(19) United States
(12) Patent Application Publication
(10) Pub. No.: US 2022/0399087 A1
(43) Pub. Date: Dec. 15, 2022

(54) METHOD AND SYSTEM FOR IMPROVED MANAGEMENT OF GENETIC DISEASES

(71) Applicants: Rady Children's Hospital Research Center, San Diego, CA (US); Alexion Pharmaceuticals, Inc., Boston, MA (US)

(72) Inventors: Stephen Kingsmore, San Diego, CA (US); Narayanan Veeraraghavan, San Diego, CA (US); Sebastien Lefebvre, Newton, MA (US)

(21) Appl. No.: 17/838,115

(22) Filed: Jun. 10, 2022

Related U.S. Application Data
(60) Provisional application No. 63/209,797, filed on Jun. 11, 2021.

(51) Int. Cl. G16H 10/60; G16B 20/20; G16H 50/20; G16H 15/00

(52) U.S. Cl. CPC G16H 10/60; G16B 20/20; G16H 50/20; G16H 15/00

(57) ABSTRACT

The present disclosure provides a method for genetic analysis for disease diagnoses, as well as a system for implementing such analysis. Provided is a comprehensive, scalable, biotechnology solution that solves diagnostic and therapeutic complications in rapidly progressive childhood genetic diseases. As such, the invention provides Genome-to-Treatment (GTRx℠), which is an automated, virtual system for genetic disease diagnosis and acute management guidance.

### CROSS-REFERENCE TO RELATED APPLICATION(S)

This application claims benefit of priority under 35 U.S.C. § 119(e) of U.S. Provisional Patent Application Ser. No. 63/209,797, filed Jun. 11, 2021. The disclosure of the prior application is considered part of and is incorporated by reference in the disclosure of this application.

### BACKGROUND OF THE INVENTION

### Field of the Invention

The invention relates generally to targeted or precision treatment of genetic disease and more specifically to a method and system for early transition from symptom-based treatment to optimal, etiology-informed management of genetic disease.

### Background Information

Collectively, the 7,103 known genetic disorders engender a large proportion of pediatric morbidity and mortality, particularly in neonatal, pediatric, and cardiovascular ICUs.¹⁻⁷ Of 140 million children worldwide suffering from rare genetic diseases, it is estimated ˜30% will not survive to their fifth birthday. In ICU settings, progression of childhood genetic diseases is often extremely rapid leading to morbidity and/or early death without a timely diagnosis and treatment. An initial, comprehensive technological solution to this problem was rapid diagnostic whole genome sequencing (rWGS®), which enabled concomitant diagnostic evaluation of almost all genetic diseases in as little as 19.5 hours. rWGS® is now being implemented nationally for inpatient diagnosis of childhood genetic disease in England, Wales, Germany, in Medicaid beneficiaries in Michigan and California, and in Anthem/Blue Cross/Blue Shield beneficiaries nationwide.

As is often true in biotechnology, rWGS® removed one bottleneck, but exposed another downstream—delayed, variable, or absent implementation of optimal, specific treatments. Clinical trials of rWGS® have identified several factors that contribute to the gap between expected and observed clinical utility of genetic disease diagnoses: Firstly, exponential advances in genomics have outpaced medical education. Most healthcare providers lack adequate genomic literacy to practice genomic medicine, and depend upon other subspecialists, particularly medical geneticists, for translation of genome reports into treatment recommendations. Geographic distance to specialty centers correlates with time to diagnosis, receipt of specialty care, and outcomes in childhood genetic diseases. In quaternary hospitals, subspecialty and superspecialty consultation leads to delays in optimal treatment. In front-line settings, lack of a full complement of subspecialists greatly limits the clinical utility of rWGS®. Secondly, many genetic diseases were either discovered only recently, or are ultra-rare, and therefore evidence-based treatment guidelines have not yet been developed. Management strategies are often interspersed across the literature in the form of case reports, case series or small cohort studies, and their relative effectiveness may not have been adjudicated. Information resources pertaining to management of rare genetic diseases are incomplete, lack interoperability, and are typically not targeted toward acute ICU treatment or front-line physicians. Upon receipt of an rWGS®-based diagnosis, these factors put an unsupportable burden on front-line physicians to search and synthesize the available treatment evidence for rare genetic diseases, many of which they may have never encountered previously. As genetic diseases are discovered, and effective, n-of-few, genetic therapies proliferate, therapeutic unfamiliarity and unwarranted variation in clinical practice will increase. Thirdly, failure to order rWGS® as a first-tier test frequently leads to diagnosis at time of hospital discharge, when management plans have been solidified or, for rapidly progressive diseases, too late to have full clinical utility.

More advanced methods are needed for clinical diagnosis of rare genetic diseases with automated provisional diagnosis as described herein.

### SUMMARY OF THE INVENTION

The present invention provides a method and autonomous system for conducting genetic analysis. The invention provides for rapid diagnosis of genetic disease.

Accordingly, in one embodiment the invention provides a method for conducting genetic analysis. The method includes:

a) determining a phenome of a subject from an electronic medical record (EMR), wherein the phenome includes a plurality of clinical phenotypes extracted from the EMR;

b) translating the clinical phenotypes into standardized vocabulary or vocabularies;

c) generating a first list of potential differential diagnoses of the subject;

d) performing genetic sequencing of a DNA sample from the subject;

e) determining genetic variants of the DNA;

f) analyzing the results of (c) and (e) to generate a second list of potential differential diagnoses of the subject, the second list being rank ordered;

g) determining the efficacy and/or quality of evidence of efficacy of available treatments for the second list of potential differential diagnoses;

h) analyzing the results of (f) and (g) to generate a third list of potential differential diagnoses of the subject, the third list being rank ordered, together with available treatments; and

k) generating a report comprising results of any of (a)-(h).

In some aspects, the method further includes: j) determining the availability of confirmatory tests for the third list of potential differential diagnoses.

In some aspects, the method further includes: k) analyzing the results of (g) and (h) to generate a fourth list of potential differential diagnoses of the subject, the fourth list being rank ordered, together with available confirmatory tests.

In aspects, the method further includes generating the EMR for the subject prior to determining the phenome of the subject. In certain aspects, translating the clinical phenotypes into standardized vocabulary is performed by extraction of phenotypes by clinical natural language processing (CNLP) and then translation into one or more standardized vocabularies. In some aspects, genetic sequencing includes rWGS®, rapid whole exome sequencing (rWES), or rapid gene panel sequencing.

In another embodiment, the invention provides a system for performing the method of the invention. The system includes a controller having at least one processor and non-transitory memory. The controller is configured to perform one or more of the processes of the method as described herein.

### BRIEF DESCRIPTION OF THE DRAWINGS

FIGS. 1A-1B depicts flow diagrams of the diagnosis of genetic diseases by standard and rapid genome sequencing. FIG. 1A is a flow diagram of the diagnosis of genetic diseases. FIG. 1B is a flow diagram of the diagnosis of genetic diseases.

FIGS. 2A-2B depicts diagrams showing clinical natural language processing can extract a more detailed phenome than manual electronic health record (EHR) review or Online Mendelian Inheritance in Man™ (OMIM™) clinical synopsis. FIG. 2A is a schematic diagram. FIG. 2B is a schematic diagram.

FIGS. 3A-3H depicts a comparison of observed and expected phenotypic features of children with suspected genetic diseases. FIG. 3A is a graphical diagram depicting data.

FIG. 3B is a graphical diagram depicting data. FIG. 3C is a graphical diagram depicting data. FIG. 3D is a Venn diagram depicting data. FIG. 3E is a graphical diagram depicting data. FIG. 3F is a graphical diagram depicting data. FIG. 3G is a graphical diagram depicting data. FIG. 3H is a Venn diagram depicting data.

FIG. 4 is a Venn diagram showing overlap of observed and expected patient phenotypic features in 95 children diagnosed with 97 genetic diseases.

FIGS. 5A-5B is a series of graphs depicting precision, recall, and F1-score of phenotypic features identified manually, by CNLP, and OMIM™. FIG. 5A is a series of graphical diagrams depicting data. FIG. 5B is a series of graphical diagrams depicting data.

FIG. 6 is a flow diagram illustrating the software components of the autonomous system and methodology for provisional diagnosis of genetic diseases by rapid genome sequencing in one aspect of the invention.

FIG. 7 is a flow diagram illustrating the software components of the autonomous system and methodology for provisional diagnosis of genetic diseases by rapid genome sequencing in one aspect of the invention.

FIGS. 8A-8B is a flow diagram of the technological components of a 13.5-hour system for automated diagnosis and virtual acute management guidance of genetic diseases by rWGS® in an aspect of the invention. FIG. 8A is a flow diagram showing the order and duration of laboratory steps and technologies. FIG. 8B is a flow diagram showing the information flow from order placement in the EHR to return of diagnostic results together with specific management guidance for that genetic disease.

FIG. 9 is a flow diagram illustrating the development of Genome-To-Treatment (GTRx℠), a virtual system for acute management guidance for rare genetic diseases.

FIGS. 10A-10B illustrates GTRx℠ disease, gene, and literature filtering, and final content. FIG. 10A is a modified PRISMA flowchart showing filtering steps and summarizing results of review of 563 unique disease-gene dyads herein. FIG. 10B is a diagram showing genetic disease types and disease genes featured in the first 100 GTRx℠ genes reviewed herein.

FIGS. 11A-11D depicts data derived using the system and methodology of the present invention. FIG. 11A shows clinical timeline of a patient. FIG. 11B shows diagnostic timeline of a patient. FIG. 11C shows clinical timeline of a patient. FIG. 11D shows diagnostic timeline of a patient.

FIG. 12 is a graphical plot depicting data pertaining to genetic sequencing costs.

### DETAILED DESCRIPTION OF THE INVENTION

The present invention is based on an innovative computational method and platform for genomic analysis. Described herein is a comprehensive, scalable, biotechnology solution to the Scylla and Charybdis of diagnostic and therapeutic odysseys in rapidly progressive childhood genetic diseases. As such, the invention provides Genome-to-Treatment (GTRx℠), also referred to herein as the system or platform of the invention, which is an automated, virtual system for genetic disease diagnosis and acute management guidance.

As discussed in detail in the Examples, by informing timely targeted treatments, rapid genetic or genomic sequencing can improve the outcomes of seriously ill children with genetic diseases, particularly infants in neonatal and pediatric intensive care units (ICUs). The need for highly qualified professionals to decipher results, however, precludes widespread implementation.

In various aspects, the present disclosure provides a platform for population-scale, provisional diagnosis of genetic diseases with automated phenotyping and interpretation. While many genetic diseases have effective treatments, they frequently progress rapidly to severe morbidity or mortality if those treatments are not implemented immediately. Since front-line physicians frequently lack familiarity with these diseases, timely molecular diagnosis may not improve outcomes. The present invention described herein is an automated, virtual system for genetic disease diagnosis and acute management guidance. Diagnosis is achieved in 13.5 hours by expedited whole genome sequencing, with superior analytic performance for structural and copy number variants. An expert panel adjudicated the indications, contraindications, efficacy, and evidence-of-efficacy of 9,911 drug, device, dietary, and surgical interventions for 563 severe, childhood, genetic diseases. The 421 (75%) diseases and 1,527 (15%) effective interventions retained are integrated with 13 genetic disease information resources and appended to diagnostic reports. This system provided correct diagnoses in four retrospectively and two prospectively tested infants. The present invention provides optimal outcomes in children with rapidly progressive genetic diseases.

Before the present compositions and methods are described, it is to be understood that this invention is not limited to the particular systems and methods described, as such systems and methods may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Methods

In one embodiment the invention provides a method for conducting genetic analysis. The analysis may be utilized to diagnose a disease or disorder, in particular a rare genetic disease. The method can also be utilized to rule out a genetic disease. The method of the invention is particularly useful in detecting and/or diagnosing a genetic disease in a subject that is less than 5 years old, such as an infant, neonate or fetus.

In some aspects the method includes:

a) determining a phenome of a subject from an electronic medical record (EMR), wherein the phenome includes a plurality of clinical phenotypes extracted from the EMR;

b) translating the clinical phenotypes into standardized vocabulary or vocabularies;

c) generating a first list of potential differential diagnoses of the subject;

d) performing genetic sequencing of a DNA sample from the subject;

e) determining genetic variants of the DNA;

f) analyzing the results of (c) and (e) to generate a second list of potential differential diagnoses of the subject, the second list being rank ordered;

g) determining the efficacy and/or quality of evidence of efficacy of available treatments for the second list of potential differential diagnoses;

h) analyzing the results of (f) and (g) to generate a third list of potential differential diagnoses of the subject, the third list being rank ordered, together with available treatments; and

k) generating a report comprising results of any of (a)-(h).

In some aspects, the method further includes: j) determining the availability of confirmatory tests for the third list of potential differential diagnoses.

In some aspects, the method further includes: k) analyzing the results of (g) and (h) to generate a fourth list of potential differential diagnoses of the subject, the fourth list being rank ordered, together with available confirmatory tests.

In some aspects, the method may further include generating the EMR for the subject prior to determining the phenome of the subject.

As used herein, “phenome” refers to the set of all phenotypes expressed by a cell, tissue, organ, organism, or species. The phenome represents an organisms' phenotypic traits.

As used herein, “EMR” refers to an electronic medical record and is used synonymously herein with “electronic health record” or “EHR”.

The method includes determining a phenome of a subject from an electronic medical record (EMR). This is performed by extracting a plurality of clinical phenotypes from the EMR. Natural language processing and/or automated feature extraction from non-standardized and standardized fields of the EMR of a subject is used to create a list of the clinical features of disease in that individual.

Translating the clinical phenotypes into standardized vocabulary is then performed utilizing a variety of computation methods known in the art. In one aspect, translation is performed by natural language processing. This type of processing is utilized for translation and mining of non-structured text. Alternatively, data organized in discrete or structured fields may be retrieved/translated utilizing a conventional query language known in the art. Embodiments of standardized vocabularies include the Human Phenotype Ontology, Systematized Nomenclature of Medicine—Clinical Terms, and International Classification of Diseases—Clinical Modification.

The method also entails generating a series of lists (e.g., first, second, third, fourth, and the like) of potential differential diagnoses of the subject. In some aspects, the method entails generating a first list of potential differential diagnoses. This is performed by query of a database populated with known clinical phenotypes expressed in the same vocabulary as the standardized vocabulary of the translated clinical phenotypes. Embodiments of databases of known clinical phenotypes include Online Mendelian Inheritance in Man™, Clinical Synopsis™, and Orphanet™ Clinical Signs and Symptoms. The list may be generated with an algorithm that rank orders all potential differential diagnoses based on goodness of fit. The list may also be generated with an algorithm that rank orders all potential differential diagnoses based on the sum of the distances of the observed and expected phenotypes in the standardized, hierarchical vocabulary.

Genetic variants are then determined from genomic sequencing performed on a DNA sample from the subject. In some aspects, this includes annotation and classification of the genetic variants. Annotation of all, or some, of the genetic variations in the subject's genome is performed to identify all variants that are of categories such as uncertain significance (VUS), pathogenic (P) or likely pathogenic (LP) and to retain genetic variations with an allele frequency of <5, 4, 3, 2, 1, 0.5, or 0.1% in a population of healthy individuals. The method may further include annotation of the genetic variants to identify and rank all diplotypes categorically, for example as being of uncertain significance (VUS), pathogenic (P) or likely pathogenic (LP) on the basis of pathogenicity. An embodiment of the classification system is the Joint Consensus Recommendation of the American College of Medical Genetics and Genomics and the Association for Molecular Pathology Standards and Guidelines for the Interpretation of Sequence Variants. The method may further include annotation of the pathogenicity of variants and diplotypes on a continuous, probabilistic scale, where a variant that is well established to be benign, for example, has a score of zero, and a variant that is well established to be pathogenic variant has a score of one, and likely benign, variants of uncertain significance, and likely pathogenic variants have scores between zero and one.

A second list of potential differential diagnoses of the subject is then generated by comparing the annotated VUS, LP and P diplotypes on a regional genomic basis with corresponding genomic regions associated with the first list of potential differential diagnoses. Genetic variants are ranked based on a combination of rank of goodness of fit of clinical phenotypes, rank of pathogenicity of diplotypes, and/or allele frequencies of the genetic variants in a population of healthy individuals. The list of potential differential diagnoses may further include annotation of their probability of being causative of the patient's condition on a continuous scale, rather than binary diagnosis/no diagnosis results.

In some aspects, the genetic variants determined from the subject's genome may be utilized to generate a probabilistic diagnosis for use in generating the second list of potential diagnoses.

A report is then generated setting forth the potential differential diagnoses of the subject, preferably in order of score to identify the diagnosis with the highest probability.

In some aspects, the method entails generating a third list, and optionally a fourth list of potential differential diagnoses. This is performed by query of a database populated with known clinical phenotypes expressed in the same vocabulary as the standardized vocabulary of the translated clinical phenotypes. Embodiments of databases of known clinical phenotypes include Online Mendelian Inheritance in Man™, Clinical Synopsis™ and Orphanet™ Clinical Signs and Symptoms. The lists may be generated with an algorithm that rank orders all potential differential diagnoses based on goodness of fit. The lists may also be generated with an algorithm that rank orders all potential differential diagnoses based on the sum of the distances of the observed and expected phenotypes in the standardized, hierarchical vocabulary.

In various aspects, the method includes determining the efficacy and/or quality of evidence of efficacy of available treatments for the list of potential differential diagnoses. In various aspects, the generated list of potential differential diagnoses of the subject, is rank order and accompanied by the suitable available treatments.

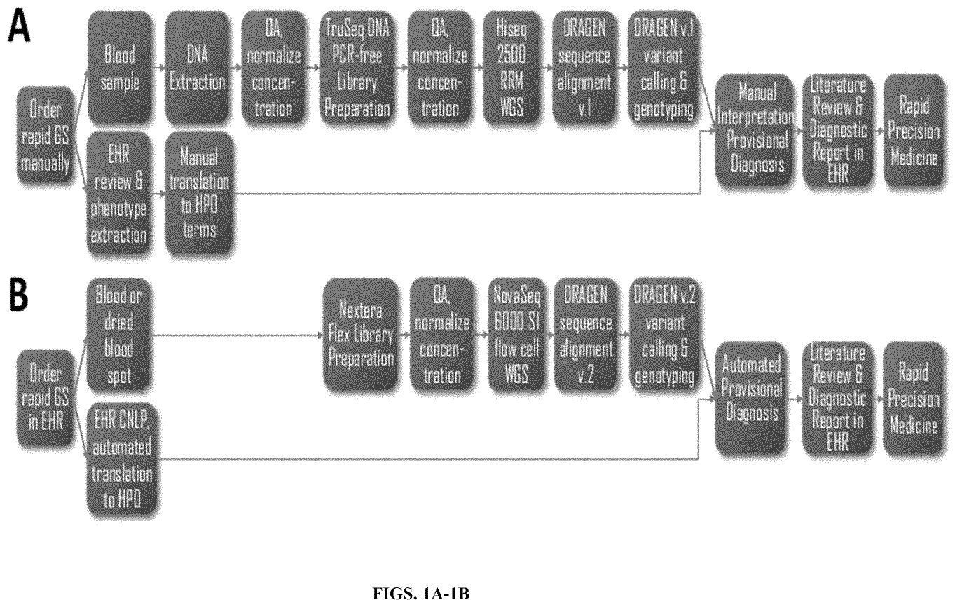

Some aspects of the invention are illustrated in FIG. 1B. FIG. 1B is a flow chart showing AI involved automated extraction of the phenome from subject's EMR by clinical natural language processing (CNLP), translation from SNOMED-CT™ to Human Phenotype Ontology™ (HPO™) terms (e.g., a standardized vocabulary), derivation of a comprehensive differential diagnosis gene list, identification of variants in genomic sequences, assembling those variants into likely pathogenic, causal diplotypes on a gene-by-gene basis, integration of the genotype and differential diagnosis lists, and retention of the highest ranking provisional diagnosis(es).

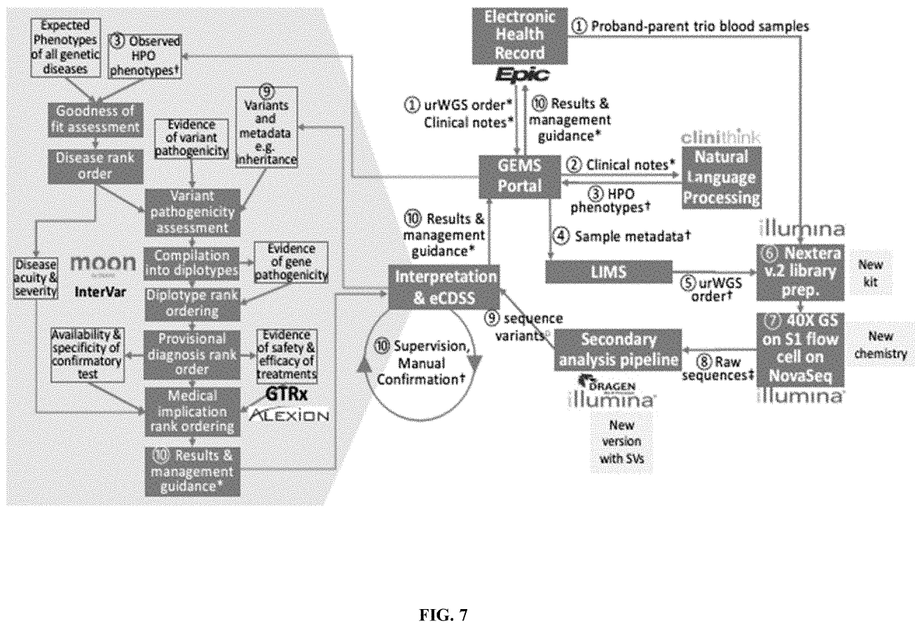

Some aspects of the invention are illustrated in FIG. 7 which is a flow diagram illustrating components of the autonomous system and methodology for diagnosis of genetic diseases by rapid genome sequencing.

The method of the present invention allows for a myriad of genetic analysis types to identify disease.

Methods described herein are useful in perinatal testing wherein the parental, e.g., maternal and/or paternal, genotypes are known. In an aspect, the methods are used to determine if a subject has inherited a deleterious combination of markers, e.g., mutations, from each parent putting the subject at risk for disease, e.g., Lesch-Nyhan syndrome. The disease may be an autosomal recessive disease, e.g., Spinal Muscular Atrophy. The disease may be X-linked, e.g., Fragile X syndrome. The disease may be a disease caused by a dominant mutation in a gene, e.g., Huntington's Disease. In some aspects, the maternal nucleic acid sequence is the reference sequence. In some aspects, the paternal nucleic acid sequence is the reference sequence. In some aspects, the marker(s), e.g., mutation(s), are common to each parent. In some aspects, the marker(s), e.g., mutation(s), are specific to one parent.

In some aspects, haplotypes of an individual, such as maternal haplotypes, paternal haplotypes, or fetal haplotypes are constructed. The haplotypes comprise alleles co-located on the same chromosome of the individual. The process is also known as “haplotype phasing” or “phasing”. A haplotype may be any combination of one or more closely linked alleles inherited as a unit. The haplotypes may comprise different combinations of genetic variants. Artifacts as small as a single nucleotide polymorphism pair can delineate a distinct haplotype. Alternatively, the results from several loci could be referred to as a haplotype. For example, a haplotype can be a set of SNPs on a single chromatid that is statistically associated to be likely to be inherited as a unit.

In some aspects, the maternal haplotype is used to distinguish between a fetal genetic variant and a maternal genetic variant, or to determine which of the two maternal chromosomal loci was inherited by the fetus.

In some aspects, the methods provided herein may be used to detect the presence or absence of a genetic variant in a region of interest in the genome of a subject, such as an infant or fetus in a pregnant woman, wherein the fetal genetic variant is an X-linked recessive genetic variant. X-linked recessive disorders arise more frequently in male fetus because males with the disorder are hemizygous for the particular genetic variant. Example X-linked recessive disorders that can be detected using the methods described herein include Duchenne muscular dystrophy, Becker's muscular dystrophy, X-linked agammaglobulinemia, hemophilia A, and hemophilia B. These X-linked recessive variants can be inherited variants or de novo variants.

In some aspects, provided herein is a method of detecting the presence or absence of a genetic variant in a region of interest in the genome of an infant or a fetus in a pregnant woman, wherein the fetal genetic variant is a de novo genetic variant or a maternally or paternally inherited genetic variant. In some aspects, the mother's and/or the father's genome is sequenced to reveal whether the genetic variant is a maternally or paternally inherited genetic variant or a de novo genetic variant. That is, if the fetal genetic variant is not present in the mother or the father, and the described method indicates that the fetal genetic variant is distinguishable from the maternal or the paternal genome, then the fetal genetic variant is a de novo variant. Accordingly, provided herein is a method of determining whether a fetal genetic variant is an inherited genetic variant or a de novo genetic variant.

In some aspects, provided herein is a method of detecting the presence or absence of a genetic variant in a region of interest in the genome of an infant or a fetus in a pregnant woman, wherein the fetal genetic variant is a de novo copy number variant (such as a copy number loss variant) or a paternally-inherited copy number variant (such as a copy number loss variant). In some aspects, the father's genome is sequenced to reveal whether the copy number variant is a paternally inherited copy number variant or a de novo copy number variant. That is, if the fetal copy number variant is not present in the father, and the described method indicates that the fetal copy number variant is distinguishable from the maternal genome, then the fetal copy number variant is a de novo copy number variant. Accordingly, provided herein is a method of determining whether a fetal copy number variant is an inherited copy number variant or a de novo copy number variant.

In some aspects, the methods provided herein allow for detecting the presence or absence of a genetic variant in a region of interest in the genome of an infant or fetus in a pregnant woman, wherein the fetal genetic variant is an autosomal recessive fetal genetic variant. In some aspects, the autosomal fetal genetic variant is an SNP. In some aspects, the fetal genetic variant is a copy number variant, such as a copy number loss variant, or a microdeletion.

In some aspects, the methods provided herein allow for detecting the presence or absence of a genetic variant that is indicative of cancer. A subject having, or suspected of having and/or developing cancer can be assessed and/or treated (e.g., by administering one or more cancer treatments to the subject). In some aspects, a cancer can be an early stage cancer. In some aspects, a cancer can be an asymptomatic cancer. A cancer can be any type of cancer. Examples of types of cancers that can be assessed and/or treated as described herein include, without limitation, lung, colorectal, prostate, breast, pancreas, bile duct, liver, CNS, stomach, esophagus, gastrointestinal stromal tumor (GIST), uterus and ovarian cancer. Additional types of cancers include, without limitation, myeloma, multiple myeloma, B-cell lymphoma, follicular lymphoma, lymphocytic leukemia, leukemia and myelogenous leukemia. In some aspects, the caner is brain or spinal cord tumor, neuroblastoma, Wilms tumor, rhabdomyosarcoma, retinoblastoma or bone cancer, such as osteosarcoma. As such, in some aspects, the cancer is a solid tumor. In some aspects, the cancer is a sarcoma, carcinoma, or lymphoma. In some aspects, the cancer is lung, colorectal, prostate, breast, pancreas, bile duct, liver, CNS, stomach, esophagus, gastrointestinal stromal tumor (GIST), uterus or ovarian cancer. In some aspects, the cancer is a hematologic cancer. In some aspects, the cancer is myeloma, multiple myeloma, B-cell lymphoma, follicular lymphoma, lymphocytic leukemia, leukemia or myelogenous leukemia.

Available treatments for a subject having, or suspected of having, cancer can be administered one or more cancer treatments. A cancer treatment can be any appropriate cancer treatment. One or more cancer treatments described herein can be administered to a subject at any appropriate frequency (e.g., once or multiple times over a period of time ranging from days to weeks). Examples of cancer treatments include, without limitation adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the subject.

The term “mutant,” “variant” or “genetic variant,” when made in reference to an allele or sequence, generally refers to an allele or sequence that does not encode the phenotype most common in a particular natural population. In some cases, a mutant allele can refer to an allele present at a lower frequency in a population relative to the wild-type allele. In some cases, a mutant allele or sequence can refer to an allele or sequence mutated from a wild-type sequence to a mutated sequence that presents a phenotype associated with a disease state and/or drug resistant state. Mutant alleles and sequences may be different from wild-type alleles and sequences by only one base but can be different up to several bases or more. The term mutant when made in reference to a gene generally refers to one or more sequence mutations in a gene, including a point mutation, a single nucleotide polymorphism (SNP), an insertion, a deletion, a substitution, a transposition, a translocation, a copy number variation, or another genetic mutation, alteration or sequence variation.

In general, the term “genetic variant” or “sequence variant” refers to any variation in sequence relative to one or more reference sequences. Typically, the variant occurs with a lower frequency than the reference sequence for a given population of individuals for whom the reference sequence is known. In some cases, the reference sequence is a single known reference sequence, such as the genomic sequence of a single individual. In some cases, the reference sequence is a consensus sequence formed by aligning multiple known sequences, such as the genomic sequence of multiple individuals serving as a reference population, or multiple sequencing reads of polynucleotides from the same individual. In some cases, the variant occurs with a low frequency in the population (also referred to as a “rare” sequence variant). For example, the variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some cases, the variant occurs with a frequency of about or less than about 0.1%. A variant can be any variation with respect to a reference sequence. A sequence variation may consist of a change in, insertion of, or deletion of a single nucleotide, or of a plurality of nucleotides (e.g. 2, 3, 4, 5, 6, 7, 8, 9, 10, or more nucleotides). Where a variant includes two or more nucleotide differences, the nucleotides that are different may be contiguous with one another, or discontinuous. Non-limiting examples of types of variants include single nucleotide polymorphisms (SNP), deletion/insertion polymorphisms (INDEL), copy number variants (CNV), loss of heterozygosity (LOH), microsatellite instability (MSI), variable number of tandem repeats (VNTR), and retrotransposon-based insertion polymorphisms. Additional examples of types of variants include those that occur within short tandem repeats (STR) and simple sequence repeats (SSR), or those occurring due to amplified fragment length polymorphisms (AFLP) or differences in epigenetic marks that can be detected (e.g. methylation differences). In some aspects, a variant can refer to a chromosome rearrangement, including but not limited to a translocation or fusion gene, or fusion of multiple genes resulting from, for example, chromothripsis.

The method of the disclosure contemplates genetic sequencing. Sequencing may be by any method known in the art. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent™ sequencing), nanopore sequencing, pyrosequencing (454), sequencing by synthesis, sequencing by ligation (SOLiD™ sequencing), sequencing by electron microscopy, dideoxy sequencing reactions (Sanger method), massively parallel sequencing, polony sequencing, and DNA nanoball sequencing. In some aspects, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some aspects, the sequencing comprises obtaining paired end reads.

In some aspects, sequencing of the nucleic acid from the sample is performed using whole genome sequencing (WGS) or rapid WGS (rWGS®). In some aspects, targeted sequencing is performed and may be either DNA or RNA sequencing. The targeted sequencing may be to a subset of the whole genome. In some aspects the targeted sequencing is to introns, exons, non-coding sequences or a combination thereof. In other aspects, targeted whole exome sequencing (WES) of the DNA from the sample is performed. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain aspects, clonally amplified DNA templates or single DNA molecules are sequenced in a massively parallel fashion within a flow cell (e.g., as described in WO 2014/015084). In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. In some aspects, the methodology of the disclosure utilizes systems such as those provided by Illumina, Inc, (HiSeq™ X10, HiSeq™ 1000, HiSeq™ 2000, HiSeq™ 2500, HiSeq™ 4000, NovaSeq™ 6000, Genome Analyzers™, MiSeq™ systems), Applied Biosystems Life Technologies (ABI PRISM™ Sequence detection systems, SOLiD™ System, Ion PGM™ Sequencer, ion Proton™ Sequencer).

In some aspects, rWGS® of DNA is performed. In some aspects, rWGS® is performed on samples of the subject, e.g., an infant, neonate or fetus. In some aspects, rWGS® is performed on maternal samples along with that of the subject. In some aspects, rWGS® is performed on paternal samples along with that of the subject. In some aspects, rWGS® is performed on maternal and paternal samples along with that of the subject.

In some aspects, rapid whole exome sequencing (rWES) of DNA is performed. In some aspects, rWES is performed on samples of the subject, e.g., an infant, neonate or fetus. In some aspects, rWES is performed on maternal samples along with that of the subject. In some aspects, rWES is performed on paternal samples along with that of the subject. In some aspects, rWES is performed on maternal and paternal samples along with that of the subject.

As used herein, the term “mutation” herein refers to a change introduced into a reference sequence, including, but not limited to, substitutions, insertions, deletions (including truncations) relative to the reference sequence. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms (SNPs), multiple nucleotide polymorphisms, insertions (e.g., insertion of one or more nucleotides at a locus but less than the entire locus), multiple nucleotide changes, deletions (e.g., deletion of one or more nucleotides at a locus), and inversions (e.g., reversal of a sequence of one or more nucleotides). The consequences of a mutation include, but are not limited to, the creation of a new character, property, function, phenotype or trait not found in the protein encoded by the reference sequence. In some aspects, the reference sequence is a parental sequence. In some aspects, the reference sequence is a reference human genome, e.g., h19. In some aspects, the reference sequence is derived from a non-cancer (or non-tumor) sequence. In some aspects, the mutation is inherited. In some aspects, the mutation is spontaneous or de novo.

As used herein, a “gene” refers to a DNA segment that is involved in producing a polypeptide and includes regions preceding and following the coding regions as well as intervening sequences (introns) between individual coding segments (exons).

The terms “polynucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. Polynucleotides may be single- or multi-stranded (e.g., single-stranded, double-stranded, and triple-helical) and contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, and the like). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may include modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may include modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, synthetic amino acids and the like), as well as other modifications known in the art.

As used herein, the term “sample” herein refers to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA. The nucleic acids in a nucleic acid sample generally serve as templates for extension of a hybridized primer. In some aspects, the biological sample is a biological fluid sample. The fluid sample can be whole blood, plasma, serum, ascites, cerebrospinal fluid, sweat, urine, tears, saliva, buccal sample, cavity rinse, feces or organ rinse. The fluid sample can be an essentially cell-free liquid sample (e.g., plasma, serum, sweat, urine, and tears). In other aspects, the biological sample is a solid biological sample, e.g., feces or tissue biopsy, e.g., a tumor biopsy. A sample can also comprise in vitro cell culture constituents (including but not limited to conditioned medium resulting from the growth of cells in cell culture medium, recombinant cells and cell components). In some aspects, the sample is a biological sample that is a mixture of nucleic acids from multiple sources, i.e., there is more than one contributor to a biological sample, e.g., two or more individuals. In one aspect, the biological sample is a dried blood spot.

In the present invention, the subject is typically a human but also can be any species with methylation marks on its genome, including, but not limited to, a dog, cat, rabbit, cow, bird, rat, horse, pig, or monkey. In one aspect, the subject is a human child. In some aspects, the child is less than 5, 4, 3, 2 or 1 year of age. In aspects, the subject is an infant, neonate or fetus.

Computer Systems

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in the medical diagnosis context, the present invention may be practiced in conjunction with any number of applications, environments and data analyses; the systems described herein are merely exemplary applications for the invention.

Methods for genetic analysis according to various aspects of the present invention may be implemented in any suitable manner, for example using a computer program operating on the computer system. An exemplary genetic analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one aspect, the computer system comprises a stand-alone system. In another aspect, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The genetic analysis system according to various aspects of the present invention and its various elements provide functions and operations to facilitate genetic analysis, such as data gathering, processing, analysis, reporting and/or diagnosis. The present genetic analysis system maintains information relating to samples and facilitates analysis and/or diagnosis. For example, in the present embodiment, the computer system executes the computer program, which may receive, store, search, analyze, and report information relating to the genome. The computer program may comprise multiple modules performing various functions or operations, such as a processing module for processing raw data and generating supplemental data and an analysis module for analyzing raw data and supplemental data to generate a disease status model and/or diagnosis information.

The procedures performed by the genetic analysis system may comprise any suitable processes to facilitate genetic analysis and/or disease diagnosis. In one embodiment, the genetic analysis system is configured to establish a disease status model and/or determine disease status in a patient. Determining or identifying disease status may comprise generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The genetic analysis system may also provide various additional modules and/or individual functions. For example, the genetic analysis system may also include a reporting function, for example to provide information relating to the processing and analysis functions. The genetic analysis system may also provide various administrative and management functions, such as controlling access and performing other administrative functions. The genetic analysis system may also provide clinical decision support, to assist the physician in the provision of individualized genomic or precision medicine for the analyzed patient.

The genetic analysis system suitably generates a disease status model and/or provides a diagnosis for a patient based on genomic data and/or additional subject data relating to the subject's health or well-being. The genetic data may be acquired from any suitable biological samples.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example 1

### Rapid Genome Sequencing for Genetic Disease Diagnosis

In this example, a prototypic, autonomous system for rapid diagnosis of genetic diseases in intensive care unit populations is described. It performs clinical natural language processing (CNLP) to automatically identify deep phenomes of acutely ill children from electronic medical records (EMR).

Experimental Materials and Methods

Study Design.

This study was designed to furnish training and test datasets to assist in the development of a prototypic, autonomous system for very rapid, population-scale, provisional diagnoses of genetic diseases by genomic sequencing, and separate datasets to test the analytic and diagnostic performance of the resultant system both retrospectively and prospectively. The 401 subjects analyzed herein were a convenience sample of the first symptomatic children who were enrolled in four studies that examined the diagnostic rate, time to diagnosis, clinical utility of diagnosis, outcomes, and healthcare utilization of rapid genomic sequencing at Rady Children's Hospital, San Diego, USA (ClinicalTrials.gov Identifiers: NCT03211039, NCT02917460, and NCT03385876). One of the studies was a randomized controlled trial of genome and exome sequencing (NCT03211039); the others were cohort studies. All subjects had a symptomatic illness of unknown etiology in which a genetic disorder was suspected. All subjects had a Rady Children's Hospital Epic EHR and a genomic sequence (genome or exome) that had been interpreted manually for diagnosis of a genetic disease. They included five groups, namely, 16 children tested for genetic diseases by rapid whole genome sequencing whose EHRs were used to train CNLP (Table 4), ten children with genetic diseases diagnosed by rapid genomic sequencing whose EHRs were used to test the performance of CNLP (Table 5), 101 children with genetic diseases diagnosed by rapid genomic sequencing whose genomic sequences and EHRs were used to test the retrospective performance of the autonomous diagnostic system, seven seriously ill children with suspected genetic diseases whose DNA samples and EHRs were used to test the prospective performance of the autonomous diagnostic system (Table 1), and 274 control children in whom rapid genomic sequencing did not disclose a genetic disease diagnosis.

Standard, Clinical, Rapid Whole Genome and Exome Sequencing, Analysis and Interpretation.

Standard, clinical, rWGS® and rWES were performed in laboratories accredited by the College of American Pathologists (CAP) and certified through Clinical Laboratory Improvement Amendments (CLIA). Experts selected key clinical features representative of each child's illness from the Epic EHR and mapped them to genetic diagnoses with Phenomizer™ or Phenolyzer™. Trio EDTA-blood samples were obtained where possible. Genomic DNA was isolated with an EZ1 Advanced XL™ robot and the EZ1 DSP DNA™ Blood kit (Qiagen). DNA quality was assessed with the Quant-iT Picogreen dsDNA™ assay kit (ThermoFisher Scientific) using the Gemini EM Microplate Reader™ (Molecular Devices). Genomic DNA was fragmented by sonication (Covaris) and bar-coded, paired-end, PCR-free libraries were prepared for rWGS® with TruSeq DNA LT™ kits (Illumina) or Hyper kits (KAPA Biosystems). Sequencing libraries were analyzed with a Library Quantification Kit™ (KAPA Biosystems) and High Sensitivity NGS Fragment Analysis Kit™ (Advanced Analytical), respectively. Paired-end 101 nt rWGS® was performed to 45-fold coverage with Illumina HiSeq™ 2500 (rapid run mode), HiSeq™ 4000, or NovaSeq™ 6000 (S2 flow cell) instruments, as described. rWES was performed by GeneDx™. Exome enrichment was with the xGen Exome Research Panel™ v1.0 (Integrated DNA Technologies), and amplification used the Herculase II Fusion™ polymerase (Agilent). Sequences were aligned to human genome assembly GRCh37 (hg19), and variants were identified with the DRAGEN™ Platform (v.2.5.1, Illumina, San Diego). Structural variants were identified with Manta™ and CNVnator™ (using DNAnexus™), a combination that provided the highest sensitivity and precision in 21 samples with known structural variants (Table 6). Structural variants were filtered to retain those affecting coding regions of known disease genes and with allele frequencies<2% in the RCIGM database. Nucleotide and structural variants were annotated, analyzed, and interpreted by clinical molecular geneticists using Opal Clinical™ (Fabric Genomics), according to standard guidelines. Opal™ annotated variants with respect to pathogenicity, generated a rank ordered differential diagnosis based on the disease gene algorithm VAAST, a gene burden test, and the algorithm PHEVOR (Phenotype Driven Variant Ontological Re-ranking), which combined the observed HPO phenotype terms from patients, and re-ranked disease genes based on the phenotypic match and the gene score. Automatically generated, ranked results were manual interpreted through iterative Opal searches. Initially, variants were filtered to retain those with allele frequencies of <1% in the Exome Variant Server™, 1000 Genomes Samples™, and Exome Aggregation Consortium™ database. Variants were further filtered for de novo, recessive and dominant inheritance patterns. The evidence supporting a diagnosis was then manually evaluated by comparison with the published literature. Analysis, interpretation and reporting required an average of six hours of expert effort. If rWGS® or rWES established a provisional diagnosis for which a specific treatment was available to prevent morbidity or mortality, this was immediately conveyed to the clinical team, as described. All causative variants were confirmed by Sanger sequencing or chromosomal microarray, as appropriate. Secondary findings were not reported, but medically actionable incidental findings were reported if families consented to receiving this information.

Natural Language Processing and Phenotype Extraction.

Extraction of HPO™ terms from the EHR entailed four steps as follows.

1) Clinical records were exported from the EHR data warehouse, transformed into a compatible format (JSON) and loaded into CLiX ENRICH™.

2) A semi-automated query map was created, using HPO™ terms (and their synonyms) as the input and CLiX™ queries as the output. The HPO™ terms were passed through the CLiX™ encoding engine, resulting in creation of CLiX™ post-coordinated SNOMED™ expressions for each recognized HPO term or synonym. Where matches were not exact, manual review was used to validate the generated CLiX™ queries. Where there was no match or incorrect matches, new content was added to the Clinithink™ SNOMED™ extension and terminology files to ensure appropriate matches between phenotypes in HPO™ and those in SNOMED-CT™. This was an iterative process that resulted in a CLiX™ query set that covered 60% (7,706) of 12,786 HPO™ terms (Oct. 9, 2017 HPO™ build).

3) EHR documents containing unstructured data were passed through the CNLP engine. The natural language processing engine read the unstructured text and encoded it in structured format as post-coordinated SNOMED™ expressions as shown in the example below which corresponds to HP0007973, retinal dysplasia:

243796009|Situation with explicit context|: {408731000|Temporal context|=410511007|Current or past|, 246090004|Associated finding|=95494009|Retinal dysplasia|, 408732007|Subject relationship context|=410604004|Subject of record|, 408729009|Finding context|=410515003|Known present|}

Each SNOMED™ expression is made up of several parts, including the associated clinical finding, the temporal context, finding context and subject context all contained within the situational wrapper. Capturing fully post-coordinated SNOMED™ expressions ensures that the correct context of the clinical note is preserved. Some HPO™ phenotypes cannot be found in SNOMED™ and can only be represented using post-coordinated expressions, as shown in the following example which is the encoding of HP0008020, progressive cone dystrophy:

243796009|Situation with explicit context|: {408731000|Temporal context|=410511007|Current or past|, 246090004|Associated finding|=(312917007|Cone dystrophy|:263502005|Clinical course|=255314001|Progressive|), 408732007|Subject relationship context|=410604004|Subject of record|, 408729009|Finding context|=410515003|Known present|}

Here, an additional attribute for ‘Clinical Course’ and an appropriate value, ‘Progressive’, are used to further qualify the expression. Clinithink™ used references to these SNOMED™ expressions, linked with Boolean logic, to create the queries corresponding to HPO™ terms. Shown below is an example query for HP0008866, failure to thrive secondary to recurrent infections:

c*hp0008866_Failure_to_thrive_secondary_to_recurrent_infections (hp0008866_1_1_Failure_to_thrive_q AND hp0002719_1_1_Infection_Recurrent_q)

q-hp0008866_1_1_Failure_to_thrive_q 243796009|Situation with explicit context|: {408731000|Temporal context|=410511007|Current or past|,246090004|Associated finding|=54840006|Failure to thrive|,408732007|Subject relationship context|=410604004|Subject of record|,408729009|Finding context|=410515003|Known present|}

q-hp0002719_1_1_Infection_Recurrent_q 243796009|Situation with explicit context|: {408731000|Temporal context|=410511007|Current or past|,246090004|Associated finding|=(40733004|Infection|:263502005|Clinical course|=255227004|Recurrent|),408732007|Subject relationship context|=410604004|Subject of record|,408729009|Finding context|=410515003|Known present|}

For an encoding created from the unstructured data to trigger one of these queries, all of the components must be matched. Therefore, the encoding of a clinical note describing an affected sibling will not trigger the query since the encoding is that of family history whilst the query looks for the term in the subject of the record (e.g., the patient). Furthermore, it should be noted that some individual HPO™ synonyms generate more than one SNOMED™ expression. Therefore, each query used in the query set is a compound of often more than 2 SNOMED™ expressions. If the above constants are stripped out from each expression (the associated clinical finding, the temporal context, finding context and subject context all contained within the situational wrapper) from each expression in the query set (along with all of the associated SNOMED™ codes), the inventors can create a more readable format to show linguistically what is included in each query created by Clinithink™.

4) This encoded data was then interrogated by the CLiX™ query technology (abstraction). To trigger an HPO query, the encoded data had to either contain an exact match, or one of its logical descendants (exploiting the parent child hierarchy of the SNOMED™ ontology), resulting in a list of HPO terms for each patient.

rWGS.

Sequencing libraries were prepared from 10 μL of EDTA blood or five 3-mm punches from a Nucleic-Card Matrix™ dried blood spot (ThermoFisher) with Nextera DNA Flex Library Prep™ kits (Illumina) and five cycles of PCR, as described. For structural variant analysis, libraries were prepared by Hyper™ kits (KAPA Biosystems), as described above. Libraries were quantified with Quant-iT Picogreen dsDNA™ assays (ThermoFisher). Libraries were sequenced (2×101 nt) without indexing on the S1 FC with Novaseq™ 6000 S1 reagent kits (Illumina). Sequences were aligned to human genome assembly GRCh37 (hg19), and nucleotide variants were identified with the DRAGEN™ Platform (v.2.5.1, Illumina).

Automated Tertiary Analysis.

Automated variant interpretation was performed using MOON™ (Diploid). Data sources and versions were ClinVar™: 2018-04-29; dbNSFP™: 3.5; dbSNP™: 150; dbscSNV™: 1.1; Apollo™: 2018-07-20; Ensembl™: 37; gnomAD™: 2.0.1; HPO™: 2017-10-05; DGV™: 2016-03-01; dbVar™: 2018-06-24; MOON™: 2.0.5). MOON™ generated a list of potential provisional diagnoses by sequentially filtering and ranking variants using decision trees, Bayesian models, neural networks, and natural language processing. MOON™ was iteratively trained with thousands of prior patient samples uploaded by prior investigators. No samples analysed in this study were used in training of MOON™.

The filtering pipeline was designed to minimize false negatives. For SNV analysis, MOON™ excluded low quality and common variants (>2% in gnomAD™), and known likely benign/Benign variants in ClinVar™. Only variants in coding regions, splice site regions and known pathogenic variants in non-coding regions were retained. A disease annotation was added to the remaining variants based on a proprietary disorder model. The disorder model performs natural language processing of the genetics literature to automatically extract associations between diseases, disease genes, inheritance patterns, specific clinical features, and other metadata on an ongoing basis.

Subsequent steps included filtering on variant frequency, with variable frequency thresholds depending on the inheritance pattern of the associated disease, known pathogenicity of the variant, and typical age of onset range of the annotated disease. In family analyses (duo/trio analysis), co-segregation of the variant with the phenotype, according to autosomal dominant, autosomal recessive, X-linked dominant or X-linked recessive inheritance patterns, was taken into account. Parent-child variant segregation was not applied as a strict filter criterion, thereby also ensuring that causal mutations following non-Mendelian inheritance (eg. with incomplete penetrance) were identified in family analyses. For proband-only analyses, only variants for which the zygosity of the called variant fit the inheritance pattern of the annotated disease were retained. In a final filter step, the phenotype overlap was scored between the input HPO terms describing the patient's phenotype and known disease manifestations of the annotated disorder annotated from the published literature. Variants in genes for which the phenotype match with the annotated disease was considered too limited based on Apollo™ were removed from the analysis. The final rank of variants was based on proprietary algorithms that took phenotype match and variant effect into account. In addition, MOON™ provided all metadata supporting the pathogenicity of ranked variants. MOON™ also returned an annotated list of all rare variants (<2% in gnomAD) and carrier status for recessive disorders.

For structural variant analysis, MOON™ removed known benign SV based on the Database of Genomic Variants™ (DGV™). SVs overlapping pathogenic SVs listed in dbVar™ were retained for analysis. From the remaining variants, MOON™ discarded SV that did not overlap with coding regions of known disease genes (Apollo™). If a family analysis was performed, segregation of the SV was taken into account, although non-Mendelian inheritance patterns (for example, incomplete penetrance) were also supported. In a final filter step, only SVs for which there was phenotype overlap between the input HPO™ terms and known disease presentations of at least one of the genes affected by the SV, were retained. MOON™ then reported a ranked list of candidate SV, where ranking was mostly based on phenotype overlap.

Statistical Analysis.

To assess the complexity of phenomes associated with childhood genetic diseases, the inventors compared phenotypes identified by manual review, CNLP, and listed for each patient's diagnosis in OMIM™. All analyses were conducted in R v3.3.3. When applying CNLP to a patient's EHR, the list of HPO™ terms produced contained both terms that had an exact match to a phenotype in the clinical notes and terms that were superclasses (ancestor terms) of exact matches. The R package ontologyIndex™ v2.4 was used to load the October 2017 build of HPO™ into R and calculate the IC of each HPO™ term in the entire OMIM™ corpus. The IC for term phenotype, which reflects its clinical specificity, is given by IC(phenotype)=−log (p_{phenotype}), where p_{phenotype} was the probability of observing the exact term or one of its subclasses across all diseases in OMIM™. Since phenotypes that were extracted manually and by CNLP were restricted to subclasses of ‘Phenotypic abnormality’ (HP:0000118), OMIM™ terms that were subclasses of ‘Clinical Modifier’ (HP:0012823), ‘Frequency’ (HP:0040279), ‘Mode of inheritance’ (HP:0000005), and ‘Mortality/Aging’ (HP:0040006) were not included in the analyses. Phenotype sets were first compared visually by plotting the HPO graph for each patient with the R package hpoPlot™ v2.4 Summary statistics for outcomes of interest include the mean, standard deviation (SD), and range. Prior to testing for significant differences, outcome variables were tested for normality using the Shapiro-Wilk test. Due to deviations from normality, differences in phenotype counts and IC were evaluated with 2-sided Mann-Whitney U tests and when the data were paired, Wilcoxon signed-rank tests. Correlation was assessed with Spearman's rank correlation coefficient (rₛ). Precision and recall were given by tp/(tp+fp) and tp/(tp+fn), respectively, where tp were true positives, fp were false positives, and fn were false negatives. The number of true positives, tp, was defined in two ways. First, tp was set to the number of HPO terms that overlapped between sets of phenotypes. Second, tp was calculated based on terms that were up to one degree of separation apart within the HPO™ hierarchy (parent-child terms) between sets of phenotypes, allowing for inexact, but similar, matches. Additional graphics were produced with packages ggplot2 v 2.2.1 and eulerr v4.0.0. A significance cutoff of p<0.05 was used for all analyses.

Results

Rapid Genome Sequencing for Genetic Disease Diagnosis.

In light of the limitations of current methods of rapid genomic sequencing, the inventors developed an automated platform for rapid, high throughput, provisional diagnosis of genetic diseases with genome sequencing by automating and accelerating our conventional workflow (FIG. 1). Conventional clinical genome sequencing requires preparatory steps of manual purification of genomic DNA from blood, DNA quality assessment, normalization of DNA concentration, sequencing library preparation, and library quality assessment (FIG. 1A). Instead, the inventors manually prepared sequencing libraries directly from blood or dried blood spots using microbeads to which transposons were attached (Nextera DNA Flex Library Prep Kit™, Illumina, Inc.; FIG. 1B), as this method was both faster and less labor intensive. Of note, dried blood spots are the sample type used in mandatory newborn screening worldwide. In four timed runs with retrospective samples, manual Nextera™ library preparation from dried blood spots took a mean of 2 hours and 45 minutes, compared with at least 10 hours by conventional DNA purification and library preparation (Truseq DNA PCR-free Library Prep Kit™, Illumina, Inc.; Table 1). As with standard methods, Nextera Flex™ allowed samples to be prepared in batches and was amenable to automation with liquid-handling robots.

Following the preparatory steps, our previous method performed rapid genome sequencing with the HiSeq™ 2500 sequencer (Illumina) in rapid run mode, with one sample sequenced per sequencing instrument (˜120 gigabases (Gb) of 2×101 nt) in ˜25 hours (FIG. 1A). Here the inventors instead performed rapid genome sequencing with the NovaSeq™ 6000 sequencer and S1 flow cell (Illumina) (FIG. 1B), as this instrument was faster and less labor-intensive, requiring fewer steps to set up a sequencing run and automatically washing the instrument after a run. In four timed runs with retrospective samples, 2×101 nt genome sequencing took a mean 15:32 hours and yielded 404-537 Gb per flow cell, sufficient for 2-3 40× genome sequences (Table 1, Table 2).

Dynamic Read Analysis for GENomics™ (DRAGEN™, Illumina) is a hardware and software platform for alignment and variant calling that has been highly optimized for speed, sensitivity and accuracy. The inventors wrote scripts to automate the transfer of files from the sequencer to the DRAGEN™ platform. The DRAGEN™ platform then automatically aligned the reads to the reference genome and identified and genotyped nucleotide variants. Alignment and variant calling took a median of 1 hour for 150 Gb of paired-end 101nt sequences (primary and secondary analysis, Table 1). Analytic performance of this new method, from blood sample receipt to output of genomic variant genotypes, was similar to standard clinical methods with reference human genome samples, retrospective patient samples, and prospective patient samples, except for lower sensitivity in the detection of nucleotide insertions/deletions (Table 2, Table 3). The new method did not assess structural variations.

CNLP of Electronic Health Records (EHRs).

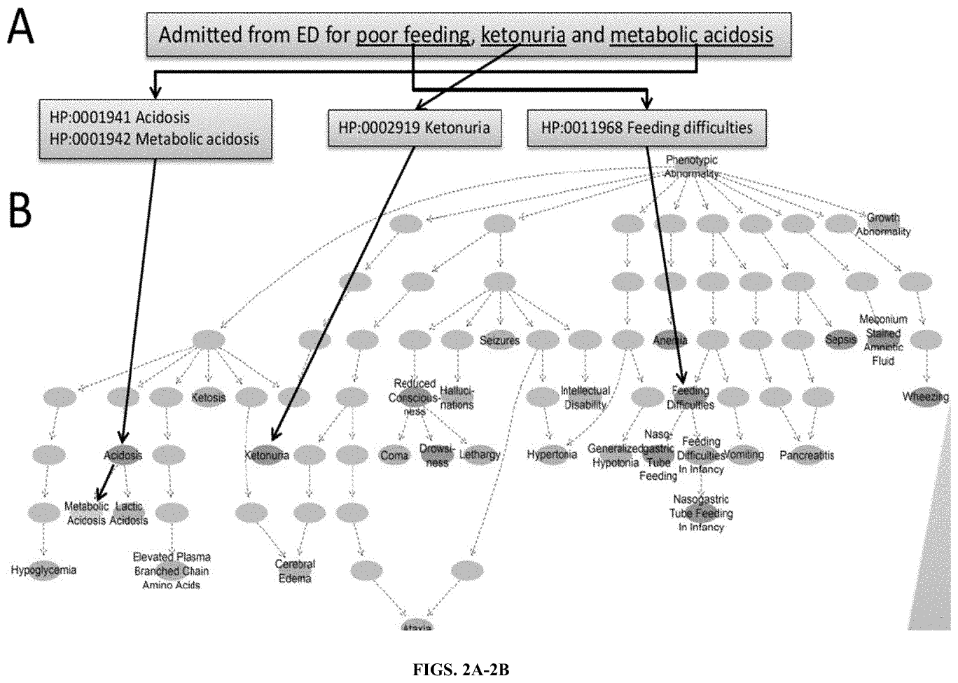

Genetic disease diagnosis requires determination of a differential diagnosis based on the overlap of the observed clinical features of a child's illness (phenotypic features) with the expected features of all genetic diseases. However, comprehensive EHR review can take hours. Additionally, manual phenotypic feature selection can be sparse and subjective, and even expert reviewers can carry an unwritten bias into interpretation (FIG. 1A). The inventors sought automated, complete phenotypic feature extraction from EHRs, unbiased by expert opinion. The simplest approach would be to extract universal, structured phenotypic features, such as International Classification of Diseases (ICD) medical diagnosis codes, or Diagnosis Related Group (DRG) codes. However, these are sparse and lack sufficient specificity. Instead, the inventors extracted clinical features from unstructured text in patient EHRs by CNLP that the inventors optimized for identification of patients with orphan diseases (CLiX ENRICH™, Clinithink™ Ltd.) (FIG. 1B, 2A). The inventors then iteratively optimized the protocol for the Rady Children's Hospital Epic EHRs using a training set of sixteen children who had received genomic sequencing for genetic disease diagnosis (Table 4). The standard output from CLiX ENRICH™ is in the form of Systematized Nomenclature of Medicine Clinical Terms (SNOMED-CT™). However, our automated methods required phenotypic features described in the Human Phenotype Ontology (HPO), a hierarchical reference vocabulary designed for description of the clinical features of genetic diseases (FIG. 2B). For this reason, the inventors mapped 7,706 (60%) of 12,786 HPO terms (13,685 including synonyms) and 75.4% of Orphanet Rare Disease HPO™ terms (June 2018 release) to SNOMED-CT™ by lexical and logical methods and then manually verified them. This enabled automated translation of phenotypic features extracted from the EHR by CNLP from SNOMED-CT™ concepts to HPO™ terms (FIG. 1B). In contrast, a previous study mapped 92% of HPO™ terms to SNOMED-CT™, but only 49% were shown to be ontologically valid and clinically relevant.

The performance of the optimized CNLP was tested with the EHRs of ten test children who had received genomic sequencing for genetic disease diagnosis. The training and test sets did not overlap. Both exact EHR phenotypic feature matches and their hierarchical root terms were extracted from first record until time of enrollment for genomic sequencing. CNLP identified a mean of 86.7 phenotypic features (standard deviation (SD) 32.8, range 26-158; Table 5) in approximately 20 seconds per patient. A detailed manual review of the EHR was performed to identify all true positive, false positive and false negative CNLP phenotypic features in the test children. Based on this, the precision (positive predictive value, PPV) of CNLP was 0.80 (SD 0.13, range 0.50-0.93) and recall (sensitivity) was 0.93 (SD 0.02, range 0.91-0.96; Table 5), which were superior to prior CNLP-based extraction of HPO terms. The principal reasons for false positives (FP) were: 1) incorrect CLiX™ encoding (n=89, 38% of 237 phenotypic features) due to misinterpreted context (n=31), unrecognized headings (n=23), incorrect acronym expansion (n=21), incorrect interpretation of a clinical word (n=8), or incorrectly attributed finding site for disease (n=6); 2) ambiguity of source text (unrecognized or incorrect syntax, abbreviations, acronyms or terminology; n=46, 19% of 237); 3) incongruity between SNOMED/HPO/clinical acumen (n=20, 8%); 4) failure to recognize a pasted citation as non-clinical text (n=68, 29%); and, 5) incorrect query logic (n=14, 6%) (Table 5).

Characterization of the CNLP-Derived Phenomes of Children with Suspected Genetic Diseases.

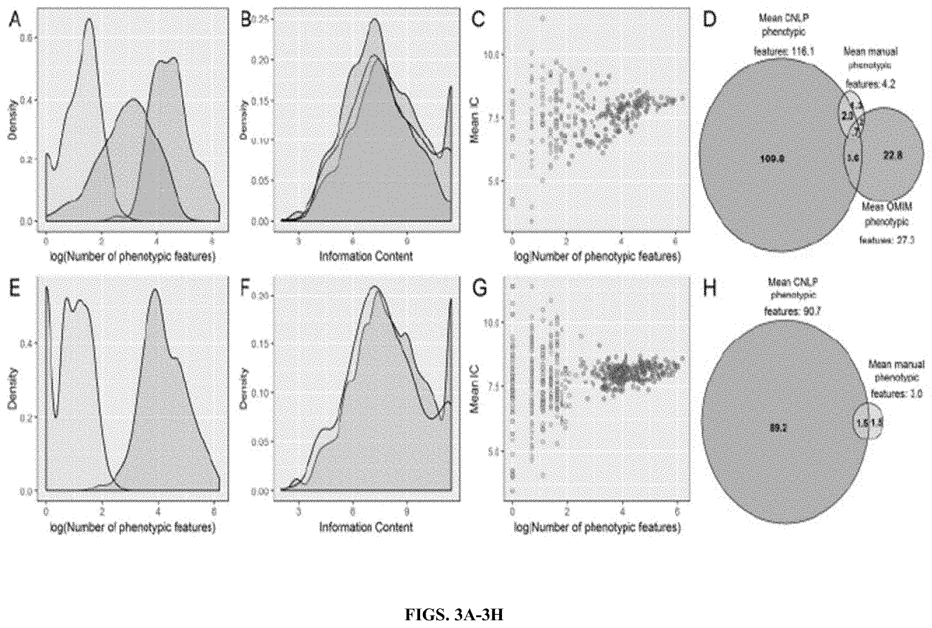

Development of an autonomous diagnostic system has been hindered by a dearth of knowledge of the topography of the phenomes of children with suspected genetic diseases. Therefore the inventors compared EHR CNLP-derived phenomes with the comparatively sparse phenotypic features selected by experts during manual interpretation of the first 375 symptomatic children to receive genomic sequencing for diagnosis of genetic diseases at Rady Children's Hospital (101 children diagnosed with genomic sequencing: FIGS. 3A-D, 274 children that were not diagnosed: FIG. 3E-H). In 101 of these children, who had received genomic diagnoses of 105 genetic diseases (four had dual diagnoses), the inventors also compared the observed phenotypic features with the expected phenotypic features for those diseases, obtained from the Clinical Synopsis field of Online Mendelian Inheritance in Man™ (OMIM™). In the 101 diagnosed children, CNLP identified 27-fold more phenotypic features (mean 116.1, SD 93.6, range 13-521) than expert manual selection at interpretation (mean 4.2, SD 2.6, range 1-16), and 4-fold more than OMIM (mean 27.3, SD 22.8, range 1-100; FIG. 3A, 3D) (45, 46). Similarly, prior studies demonstrated 2-fold more phenotypic features extracted by CNLP than comprehensive, expert manual extraction, and 18-fold more phenotypic features extracted by CNLP than Orphanet HPO™ terms for those diseases. CNLP extracted more phenotypic features in the 101 diagnosed children than the 274 undiagnosed children (mean, 116.1 vs 90.7, respectively; P=0.0004, Mann-Whitney U test; FIG. 3A, 3D, 3E, 3H). This suggested the possibility that undiagnosed children, in part, did not have enough detail in their medical records to make a molecular diagnosis. In addition, there was greater overlap between CNLP- and manually-extracted phenotypic features in diagnosed children (mean 2.74 terms, SD 1.7, range 0-9) than undiagnosed (mean 1.52 terms, SD 1.48, range 0-7; P<0.0001, Mann—Whitney U test; FIG. 3D, 3H). This suggested that undiagnosed children, in part, had less consistent information on phenotypic features.

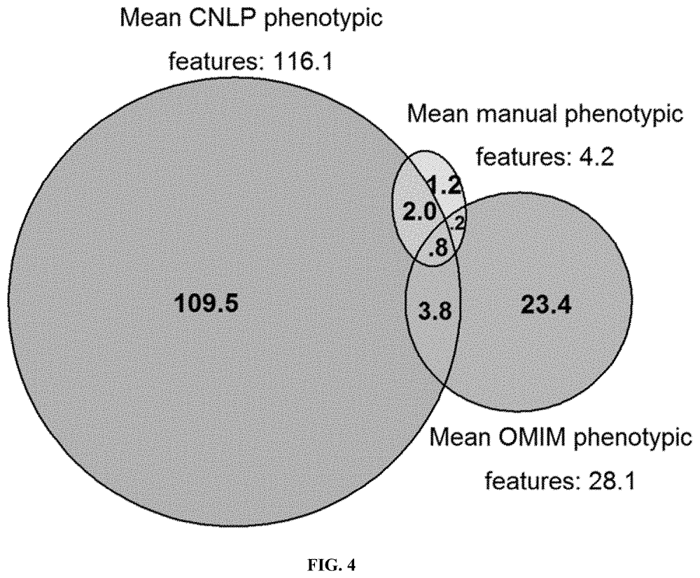

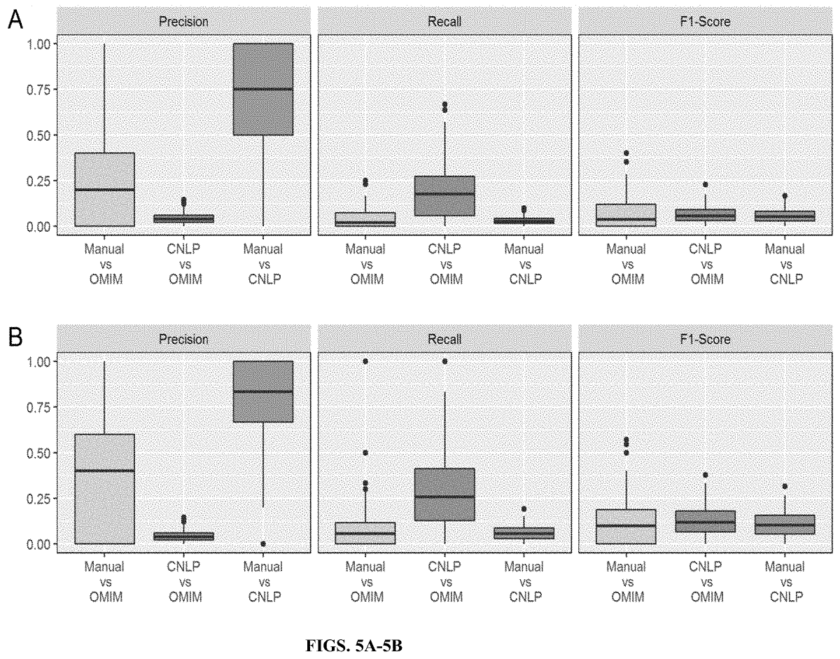

In the 101 diagnosed children, phenotypic features extracted by CNLP overlapped expected OMIM™ phenotypic features (mean 4.31 terms, SD 4.59, range 0-32) significantly more than the manual extracted phenotypic features (mean 0.92 terms, SD 1.02, range 0-4; P<0.0001, paired Wilcoxon test; FIG. 3B). Although the cohort included eight genetic diseases that were incidental findings, their exclusion did not materially change these results (FIG. 4). Thus, the recall of OMIM™ phenotypic features by CNLP, although small (mean 0.20, SD 0.16, range 0-0.67), was substantially greater than the sparse expert manual phenotypic features used in expert manual interpretation (mean 0.04, SD 0.06, range 0-0.25) (FIG. 5). However, the much larger number of phenotypic features extracted by CNLP was associated with lower precision (mean 0.04, SD 0.03, range 0-0.15) than manual extraction (mean 0.25, SD 0.30, range 0-1) when compared with OMIM™, indicating that, by design, an autonomous diagnostic system should not penalize false positive phenotypic features. Recall and F₁ value increased when phenotypic features with one degree of hierarchical separation to those extracted were included (mean CNLP recall with inexact matches 0.29, SD 0.22, range 0-1; mean CNLP F₁ with inexact matches 0.12, SD 0.08, range 0-0.38; mean CNLP F₁ with exact matches 0.06, SD 0.05, range 0-0.23), indicating that, by design, an autonomous system should include hierarchical parents of extracted terms (FIG. 5).

Traditionally, genetic diseases have been clinically diagnosed by the identification of one or more pathognomonic phenotypic features. Such phenotypic features have high information content (IC, the logarithm of the probability of that phenotypic feature being observed in all OMIM™ diseases; FIG. 2). A potential concern was that phenotypic features extracted by CNLP would have less information content than those prioritized manually by experts during interpretation. However, among the 101 children, the mean IC of CNLP phenotypic features (8.1, SD 2.0, range 2.6-11.4) was significantly higher than manual (7.8, SD 2.0, range 2.1-11.4; P=0.003, Mann-Whitney U test) or OMIM™ phenotypic features (7.3, SD 1.7, range 3.2-11.4; P<0.0001, Mann-Whitney U test, FIG. 3E). The inventors note that the mean IC correlated significantly with number of phenotypic features extracted manually and by CNLP (Spearman's rho 0.24, P=0.02 and Spearman's rho 0.44, P<0.0001, respectively; FIG. 3C). The mean IC of CNLP phenotypic features was higher than manual phenotypic features (FIG. 3F), and the mean IC correlated significantly with number of phenotypic features extracted by CNLP (Spearman's rho 0.30, P<0.0001; FIG. 3G).

Retrospective Performance of an Autonomous System for Diagnosis of Childhood Genetic Diseases.

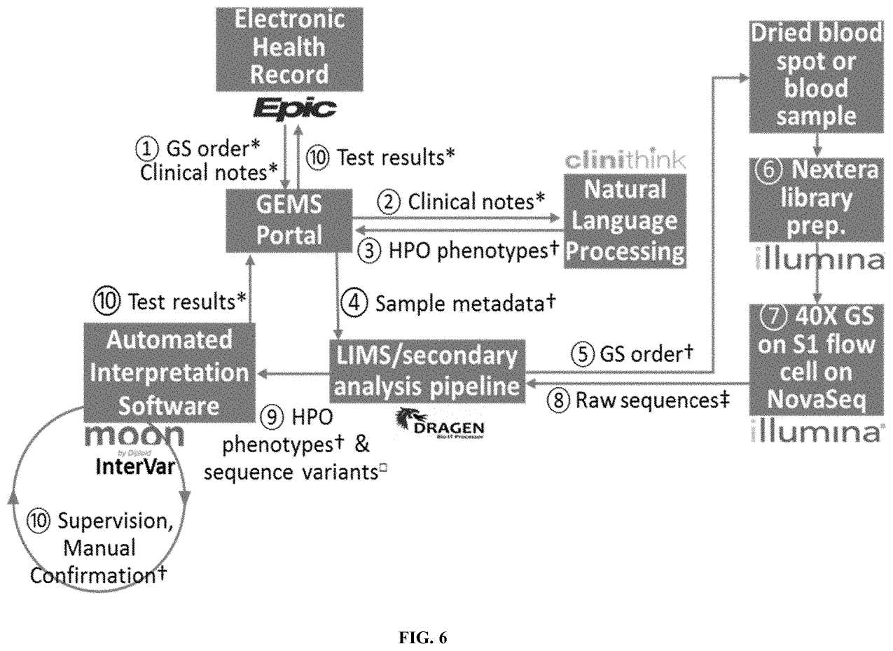

The remaining steps in automated diagnosis of genetic diseases were to combine the automated ranking of the patient's CNLP phenome with respect to all genetic diseases, together with the automated ranking of the pathogenicity of all their genomic variants based on literature knowledge and in silico tools (FIG. 1, FIG. 6). The inventors wrote scripts to transfer the patient's CNLP-derived phenotypic features and genomic variants automatically to autonomous interpretation software (MOON™, Diploid). MOON™ identified the phenotypic features associated with each genetic disease by natural language processing of the medical literature. Typically, this was a larger set of phenotypic features than those listed in the OMIM™ Clinical Synopsis. MOON™ then compared the patient's phenotypic features with those associated with each genetic disease and rank-ordered their likelihood of causing the child's illness.

The inventors also wrote scripts to transfer a patient's nucleotide and structural variants automatically from the DRAGEN™ platform to MOON™ as soon as it finished, without user intervention. For rapid genome sequencing, there was a mean of 4,742,595 nucleotide variants and 19.3 structural variants (SVs) and exome sequencing had a mean of 39,066 nucleotide variants and 10.3 SVs per patient. Of these, MOON™ retained 67,589 nucleotide variants and 12 SVs, and 791 nucleotide variants and 4.5 SVs, for rapid genome and exome sequencing, respectively, that had allele frequencies<2% and affected known disease genes. A Bayesian framework and probabilistic model in MOON™ ranked the pathogenicity of these variants with 15 in silico prediction tools, ClinVar™ assertions, and inheritance pattern-based allele frequencies. In singleton and family trio analyses, a mean of five and three provisional diagnoses were ranked, respectively (Table 6). Since MOON™ was optimized for sensitivity, it shortlisted a median of 6 nucleotide variants per diagnosed subject (range 2-24), and often shortlisted false positive diagnoses in cases considered negative by manual interpretation. Both were largely remedied, however, by processing the MOON™ output in InterVar™ software, and retaining only pathogenic and likely pathogenic variants. InterVar™ classified variants with regard to 18 of the 28 consensus pathogenicity recommendations, specifically triaging variants of uncertain significance (VUS). Automated interpretation took a median of five minutes from transfer of variants and HPO™ terms to display of the provisional diagnosis and supporting evidence, including patient phenotypic features matching that disorder, for laboratory director review. In four timed runs, the time from blood or blood spot receipt to display of the correct diagnosis as the top ranked variant was 19:14-20:25 hours (median 19:38 hours, Table 1, retrospective cases). This conformed well to a daily clinical operation cycle: sample receipt in the morning enabled library preparation in the afternoon, genome sequencing overnight, and provisional reporting early the following morning for laboratory director review.

The inventors retrospectively examined the concordance between the autonomous system and prior, team-based, manual expert interpretation in 95 of the 101 children, diagnosed with 97 of the 105 genetic diseases. The inventors excluded 8 findings that had been reported but that were considered incidental (without current evidence of any of the expected phenotypic features). This cohort was diverse in race and ancestry. Eleven diagnoses were associated with structural variants, and 86 with nucleotide variants. No training patients were included in the test set. In two patients, a revised clinical report was issued of a new diagnosis (infant 6007, EIEE9, Xp22 del, and patient 6033, Cockayne syndrome B, ERCC6 p.Gly528Glu and c.-15+3G>T, which was validated by functional studies). Therefore, initial expert manual interpretation had a recall of 98% (95 of 97). Although the inventors did not re-analyze manual diagnoses, none of them had been demoted in the period since initially reported clinically. The autonomous diagnostic system had precision of 99% (93 of 94) and recall of 97% (94 of 97). For nucleotide and structural variants, the median rank of the correct diagnosis was first (range 1-4 nucleotide variants; range 1-13 SV; Table 6).

The three false negative autonomous diagnoses comprised the following cases.

Infant 6159, with autosomal dominant Alport syndrome (COL4A4 c.4715C>T, p.Pro1572Leu), had hematuria, nephrotic syndrome, glomerulonephritis, hypertension, and anasarca. OMIM™ indicated COL4A4-associated Alport syndrome (CAS) was autosomal recessive, and p.Pro1572Leu was recorded as pathogenic in ClinVar™ for autosomal recessive Alport syndrome. There are, however, a large number of reports of autosomal dominant CAS. The variant was maternally inherited. Since the infant's mother was asymptomatic, the inventors assumed that she exhibited incomplete penetrance of autosomal dominant CAS, as has been reported. The autonomous system classified the infant as a carrier for autosomal recessive CAS.

Infant 253 had autosomal dominant optic atrophy plus syndrome (OPA1 c.556+1G>A). The autonomous system did not rank this variant because of insufficient overlap of the 70 CNLP phenotypic features with the MOON™ disease phenotypic feature model. Recent reports indicate that OPA1 can be associated with complex, severe multi-system mitochondrial disorders, similar to infant 253.

Neonate 213 had dextrocardia and transposition of the great vessels. He received singleton genome sequencing, and was diagnosed manually with autosomal dominant visceral heterotaxy type 5 associated with a likely pathogenic variant in NODAL (c.778G>A; p.Gly260Arg). This variant was filtered out by the autonomous system based on classification as a VUS by InterVar™ (based on PM1-PP3-PP5) and the presence of conflicting interpretations in ClinVar, including a ‘Likely Benign’ assertion.

When the relatively sparse phenotypic features selected by experts during manual interpretation were substituted for phenotypic features identified by CNLP, the recall of the autonomous system decreased (88%, 85 of 97).

Prospective Performance of an Autonomous System for Diagnosis of Childhood Genetic Diseases.

The inventors prospectively compared the performance of the autonomous diagnostic system with the fastest manual methods in seven seriously ill infants in intensive care units and three previously diagnosed infants (Table 1). The median time from blood sample to diagnosis with the autonomous platform was 19:56 hours (range 19:10-31:02 hours), compared with the median manual time of 48:23 hours (range 34:38-56:03 hours). This included two automated runs which were delayed by operator error or data center downtime. The autonomous system coupled with InterVar™ post-processing made three diagnoses and no false positive diagnoses. All three diagnoses were confirmed by manual methods and Sanger sequencing. The first was for patient 352, a seven-week-old female, admitted to the pediatric intensive care unit with diabetic ketoacidosis. Rapid genome sequencing was performed on the singleton proband. In 19:11 hours, the autonomous system identified a previously unreported, heterozygous missense variant in the insulin gene (INS c.26C>G, pPro9Arg), which is associated with autosomal dominant permanent neonatal diabetes mellitus (OMIM™ disease record 606176). According to ACMG/AMP pathogenicity criteria, the variant was of uncertain significance (VUS). After 42:04 hours, parent-child trio sequencing with the fastest manual methods confirmed the result and showed the variant to be de novo, which changed the variant classification to likely pathogenic.

The second diagnosis was made in patient 7052, a previously healthy 17-month-old boy admitted to the pediatric intensive care unit with pseudomonal septic shock, metabolic acidosis, echthyma gangrenosum and hypogammaglobulinemia. Singleton, proband, rapid sequencing and automated interpretation identified a pathogenic hemizygous variant in the Bruton tyrosine kinase gene (BTK c.974+2T>C) associated with X-linked agammaglobulinemia 1 (OMIM™: 300755) in 22:04 hours. This was 16:33 hours earlier than a concurrent trio run with the fastest manual methods. The provisional result provided confidence in treatment with high-dose intravenous immunoglobulin (to maintain serum IgG>600 mg/dL) and six weeks of antibiotic treatment. This provisional diagnosis was verbally conveyed to the clinical team upon review of the autonomous result by a laboratory director. Clinical whole genome sequencing subsequently returned the same result and showed the variant to be maternally inherited.

The third diagnosis was made in patient 412, a 3-day-old boy admitted to the neonatal ICU with seizures and a strong family history of infantile seizures responsive to phenobarbital. The autonomous system identified a likely pathogenic, heterozygous variant in the potassium voltage-gated channel, KQT-like subfamily, member 2 gene (KCNQ2 c.1051C>G). This gene is associated with autosomal dominant benign familial neonatal seizures 1 (OMIM™ disease record 121200). The diagnosis was made in 20:53 hours, which was 27:30 hours earlier than a concurrent run with the fastest manual methods. A verbal provisional result was conveyed to the clinical team upon review of the result by a laboratory director as the diagnosis provided confidence in treatment with phenobarbital and changed the prognosis.

For the remaining four patients, no diagnosis was evident with either manual or autonomous methods.

Discussion

Previously, the fastest time to diagnosis by genome sequencing in clinical practice was 37 hours. The protocol was, however, extremely labor- and capital-intensive, and limited to one sample at a time. Here the inventors described a prototypic, autonomous system for genetic disease diagnosis in a median of 20:10 hours requiring decreased user intervention and a throughput of up to two parent-child trios or six probands per run. Most decision making in ICUs is made deliberatively in morning rounds attended by a multidisciplinary healthcare team. Thus, a 20-hour diagnosis would return results to the on-call physician who ordered testing in time for morning rounds. This would simplify information transfer during rounds and facilitate management decisions. A 20-hour diagnosis is important in seriously ill infants as a majority of timely genomic diagnoses result in changes in ICU management.

The autonomous platform for 20-hour diagnosis of genetic diseases was designed to meet the needs of acutely ill infants in ICUs with diseases of unknown etiology. It has been estimated that 10-12% of infants admitted to regional ICUs may benefit from same-day diagnosis and implementation of targeted treatments. In 2014, the US Food and Drug Administration (FDA) permitted provisional reporting in seriously ill children when the diagnosis indicated changes in management that could improve outcome, and where a delay in reporting until confirmation of results by Sanger sequencing could result in avoidable morbidity or mortality. In our previous experience, provisional diagnoses were reported in 17% (114 of 684) of genome sequencing cases, with a mean time to report of 3.6 days. Presentations in which 20-hour diagnoses were likely to be associated with improved outcomes included neonatal epileptic encephalopathies, metabolic diseases (as in patient 352), septic shock possibly associated with immunodeficiency (as in patient 7052), organ failure, and when extra-corporeal membrane oxygenation is considered in the absence of a known disease etiology. Thus, a circumscribed application of an autonomous diagnostic system is to identify provisional diagnoses for laboratory director review, earlier than standard rapid testing, in a subset of neonatal and pediatric ICU admissions in which morbidity or mortality is likely to be avoided by early institution of targeted treatment. It will be important to evaluate the proportion of seriously ill patients and extent of urgent healthcare settings in which a 20-hour diagnosis would inform acute interventions and for which a longer time to result would not be effective.

This disclosure demonstrated the automated extraction of a deep, digital phenome from the EHR. The analytic performance of the extraction of phenotypic features from the EHRs of children with genetic diseases by CNLP herein was considerably better than prior reports, and appeared adequate for replacement of expert manual EHR review. CNLP extracted 27-fold more phenotypic features from the EHR than those selected by experts during manual interpretation, consistent with prior reports. In addition, the mean information content of the CNLP phenome was greater than that of the phenotypic features selected by experts during manual interpretation. The superiority of deep CNLP phenomes was shown by substantially greater overlap with the expected (OMIM™) clinical features than by those selected by experts during manual interpretation. Phenotypic features selected by experts during manual interpretation had poorer diagnostic utility than CNLP-based phenotypic features when used in the autonomous diagnostic system. This concurred with two recent reports of genomic sequencing of cohorts of patients in which the rate of diagnosis was greater when more than fifteen phenotypic features were used at time of interpretation that when one to five were used.

Herein the inventors described fully automated interpretation of sequencing results. In 95 seriously ill children, the autonomous system had 97% recall and 99% precision in recapitulating 97 genetic disease diagnoses made by a team of experts. Where the system suggested more than one diagnosis, the median rank of a variant associated with the correct diagnosis was first. The three false negative autonomous results had explanations that either can be addressed by parameter adjustments or were of types that cause assessments of variant pathogenicity to vary between laboratories. Prospectively, molecular laboratory directors determined that the autonomous system made correct provisional diagnoses in three of seven seriously ill ICU infants (100% precision and recall) with an average time saving of 22:19 hours. In light of insufficient expert analysts, molecular laboratory directors, medical geneticists and genetic counselors to expand genomic diagnosis to regional ICU infants worldwide, such diagnostic performance was sufficient to suggest several, high throughput clinical applications. Supervised autonomous systems may provide effective first-tier, provisional diagnoses, allowing valuable cognitive resources to be reserved for unsolved or difficult cases, manual curation of variants, and clinical report generation which includes a summary of medical management literature. Secondly, in the roughly 67% of cases where manual interpretation fails to provide a diagnosis, it is difficult to know when analysis should be considered complete. With further development, autonomous diagnostic systems could provide an independent, objective analysis in such cases. Thirdly, autonomous systems could re-analyze unsolved cases periodically. This is burdensome to perform manually since 250 new gene-disease associations and 9,200 new variant-disease associations are reported annually. However, re-analysis yields up to 8-10% new diagnoses per annum. Automated re-analysis could include updated CNLP of the EHR, which would useful when the phenotype evolves with time. A known risk of genetic testing is over-treatment as a result of over-diagnosis. Periodic, autonomous re-analysis would also detect cases where the diagnosis is changed as a result of reclassification of the causality of the gene or pathogenicity of the variant and/or phenome overlap was minimal. An autonomous system, akin to an autopilot, can decrease the labor intensity of genome interpretation. 106 years after the invention of the autopilot, however, two pilots are still employed in cockpits of commercial aircraft. Likewise, a skilled team will still be required to curate the literature and make tough decisions/classifications for the foreseeable future.

The autonomous system has several limitations. Firstly, system performance is partly predicated on the quality of the history and physical examination, and completeness of the write-up in EHR notes. The performance of the autonomous diagnostic system, though acceptable, is anticipated to improve with additional training, increased mapping of human phenotype ontology terms associated with genetic diseases in OMIM™, Orphanet™ and the literature to SNOMED-CT™, the native language of the CNLP, inclusion of phenotypes from structured EHR fields, measurements of phenotype severity (such as phenotype term frequency in EHR documents), and material negative phenotypes (pathognomonic phenotypes whose absence rules out a specific diagnosis). As part of this, a quantitative data model is needed for improved multivariate matching of non-independent phenotypes that appropriately weights related, inexact phenotype matches. Although possible, the autonomous system did not take advantage of commercial variant database annotations, such as the Human Gene Mutation Database™, and does not eliminate the labor-intensive literature curation which is the current standard for variant reporting. Diagnosis of genetic diseases due to structural variants requires standard library preparation and additional software steps that add several hours to turnaround time. Because the autonomous system utilizes the same knowledge of allele and disease frequencies as manual interpretation, which under-represent minority races or ethnicities, pathogenicity assertions in the latter groups are less certain. Likewise, as the autonomous system utilizes the same consensus guidelines for variant pathogenicity determination as manual interpretation, it is subject to the same general limitations of assertions of pathogenicity.

The major barriers to widespread adoption of genomic medicine for seriously ill infants with disorders of unknown etiology are an untrained medical workforce and substantial shortage of domain experts, including medical geneticists, molecular laboratory directors and genetic counselors. Manual genome analysis and interpretation are very labor intensive. In addition, the extreme number of rare genetic diseases precludes easy domain mastery by non-experts. Thus, pediatric genomic medicine may be one of the first clinical areas where artificial intelligence is necessary for its general adoption. Diagnosis of seriously ill infants with diseases of unknown etiology represents an early application of autonomous diagnostic systems as such cases are abundant in ICUs and a faster time to result is critical for optimal outcomes.

### Figure Legends

FIG. 1. Flow diagrams of the diagnosis of genetic diseases by standard and rapid genome sequencing. A. Steps in conventional clinical diagnosis of a single patient by genome sequencing (GS) with manual analysis and interpretation in a minimum of 26 hours, but with mean time-to-diagnosis of sixteen days (8, 16-30). Genome sequencing was requested manually. The inventors extracted genomic DNA manually from blood, assessed DNA quality (QA), and normalized the DNA concentration manually. The inventors then manually prepared TruSeq PCR-free DNA™ sequencing libraries, performed QA again, and normalized the library concentration manually. Genome sequencing was performed on the HiSeq™ 2500 system (Illumina) in rapid run mode (RRM). Sequences were manually transferred to the DRAGEN™ Platform version 1 (Illumina) for alignment and variant calling. Phenotypic features were identified by manual review of the electronic health record (EHR). Variant files and phenotypic features were loaded manually into Opal™ software (Fabric), and interpretation was performed manually. B. Steps in autonomous diagnosis of up to six patients concurrently in a minimum of 19 hours (FIG. 6). Steps included: 1. Automation of order entry from the EHR with a portal; 2. Manual or robotic preparation of Nextera DNA Flex™ sequencing libraries directly from blood in 2.5 hours; 3. Rapid 40-fold coverage genome sequencing in 15.5 hours with the NovaSeq 6000 system and S1 flowcell (Illumina); 4. Automation of sequence transfer, alignment and variant calling in one hour with the DRAGEN platform, version 2 (Illumina); 5. Automated extraction of patient phenomes from the EHR by clinical natural language processing (CNLP), and translation to human phenotype ontology (HPO) terms in 20 seconds; 6. Automated transfer of variant and phenotype files, and automated Bayesian comparison of the CNLP phenome with those of all genetic diseases (MOON, Diploid), combined with automated assessment of the pathogenicity of their genomic variants based on aggregated literature knowledge and in silico predictive tools (InterVar) and automated display of the highest ranked provisional diagnosis(es).

FIG. 2. Clinical natural language processing can extract a more detailed phenome than manual EHR review or OMIM™ clinical synopsis. A. Example CNLP of a sentence from the EHR of an eight-day-old baby (patient 341) with maple syrup urine disease, showing four extracted HPO terms. B. Hierarchical display of HPO phenotypic features extracted by manual review of the EHR of neonate 341, CNLP (red), and expected phenotypic features (from the OMIM™ Clinical Synopsis, blue). Yellow circles: Phenotypic features extracted by both CNLP and expert review. Purple circles: Phenotypic overlap between CNLP and OMIM™. Grey circles: The location of parent terms of identified phenotypic features within the HPO hierarchy. The Information Content (IC) was defined by IC (phenotype)=−log (p_{phenotype}), where p_{phenotype} was the probability of observing the exact term or one of its subclasses across all diseases in OMIM™. Information content increases from top (general) to bottom (specific).

FIG. 3. Comparison of observed and expected phenotypic features of 375 children with suspected genetic diseases. A-D: 101 children diagnosed with 105 genetic diseases. E-H: 274 children with suspected genetic diseases that were not diagnosed by genomic sequencing. Phenotypic features identified by manual EHR review are in yellow, those identified by CNLP are in red, and the expected phenotypic features, derived from the OMIM™ Clinical Synopsis, are in blue. A. Frequency distribution of the number of phenotypic features (log-transformed) in 101 children with genetic diseases. The mean number of features detected per patient was 4.2 (SD 2.6, range 1-16) for manual review, 116.1 (SD 93.6, range 13-521) for CNLP, and 27.3 (SD 22.8, range 1-100) for OMIM™ (OMIM™ vs Manual: P<0.0001; CNLP vs OMIM™: P<0.0001; CNLP vs Manual: P<0.0001; paired Wilcoxon tests). B. Frequency distribution of information content (IC) for each phenotypic feature set in 101 diagnosed patients. The mean IC was 7.8 (SD 2.0, range 2.1-11.4) for manual review, 8.1 (SD 2.0, range 2.6-11.4) for CNLP, and 7.3 (SD 1.7, range 3.2-11.4) for OMIM™ (Manual vs OMIM™: P<0.0001; CNLP vs OMIM™: P<0.0001; Manual vs CNLP: P=0.003; Mann-Whitney U tests). C. Correlation of the mean information content of phenotypic terms with the number of phenotypic terms in each patient. Spearman's rank correlation coefficient (rₛ) was 0.24 for manually extracted phenotypic features (P=0.02), 0.44 for CNLP (P<0.0001) and −0.001 for OMIM™ (P>0.05). D. Venn diagram showing overlap of phenotypic terms by the three methods for diagnosed patients. Phenotypic features extracted by CNLP overlapped expected OMIM™ phenotypic features (mean 4.31 terms, SD 4.59, range 0-32) significantly more than manually (mean 0.92 terms, SD 1.02, range 0-4; P<0.0001, paired Wilcoxon test for the difference in the number of terms that overlap with OMIM™). E. Frequency distribution of the number of phenotypic features (log-transformed) in 274 children with suspected genetic diseases that were not diagnosed by genomic sequencing. The mean number of features was 3.0 (SD 1.9, range 1-12) for manual review and 90.7 (SD 81.1, range 6-482) for CNLP (CNLP vs Manual: P<0.0001, paired Wilcoxon test). F. Frequency distribution IC for each phenotypic feature set in 273 undiagnosed patients. The mean IC was 7.7 (SD 2.1, range 2.1-11.4) for manual review and 8.1 (SD 2.0, range 2.6-11.4) for CNLP (Manual—CNLP: P<0.0001, Mann-Whitney U test). G. Correlation of the mean information content of phenotypic terms with the number of phenotypic terms in each patient. rₛ was 0.02 for manually extracted phenotypic features (P>0.05) and 0.30 for CNLP (P<0.0001). H. Venn diagram showing overlap of phenotypic terms for undiagnosed patients by CNLP and manual methods.

FIG. 4. Venn diagram showing overlap of observed and expected patient phenotypic features in 95 children diagnosed with 97 genetic diseases. Phenotypic features identified by expert manual EHR review during interpretation are shown in yellow. Phenotypic features identified by CNLP are shown in red. The expected phenotypic features are derived from the OMIM™ Clinical Synopsis and are shown in blue. The inventors excluded eight diagnoses that were considered to be incidental findings. Phenotypes extracted by CNLP overlapped expected OMIM™ phenotypes (mean 4.55, SD 4.62, range 0-32) more than phenotypes that were manually extracted (mean 0.97, SD 1.03, range 0-4).

FIG. 5. Precision, recall, and F1-score of phenotypic features identified manually, by CNLP, and OMIM™. Data are from 101 children with 105 genetic diseases. Precision (PPV) was given by tp/tp+fp, where tp were true positives and fp were false positives. Recall (sensitivity) was given by tp/tp+fn, where fn were false negatives. A. Precision and recall calculated based on exact phenotypic feature matches. Manual vs OMIM™—Precision: mean 0.25, SD 0.30, range 0-1; Recall: mean 0.04, SD 0.06, range 0-0.25; F₁: mean 0.07, SD 0.09, range 0-0.40. cNLP vs OMIM™—Precision: mean 0.04, SD 0.03, range 0-0.15; Recall: mean 0.20, SD 0.16, range 0-0.67; F₁: mean 0.06, SD 0.05, range 0-0.23. Manual vs cNLP—Precision: mean 0.71, SD 0.28, range 0-1; Recall: mean 0.03, SD 0.02, range 0-0.1; F₁: mean 0.06, SD 0.04, range 0-0.17. B. Precision and recall calculated allowing for inexact phenotype matches (terms with one degree of hierarchical separation). Manual vs OMIM™—Precision: mean 0.4, SD 0.34, range 0-1; Recall: mean 0.09, SD 0.13, range 0-1; F₁: mean 0.13, SD 0.13, range 0-0.57. cNLP vs OMIM™—Precision: mean 0.09, SD 0.07, range 0-0.38; Recall: mean 0.29, SD 0.22, range 0-1; F₁: mean 0.12, SD 0.08, range 0-0.38. Manual vs cNLP—Precision: mean 0.79, SD 0.24, range 0-1; Recall: mean 0.06, SD 0.04, range 0-0.19; F₁: mean 0.11, SD 0.07, range 0-0.32.

FIG. 6. Flow diagram of the software components of the autonomous system for provisional diagnosis of genetic diseases by rapid genome sequencing. Abbreviations: GS: rapid whole genome sequencing; GEMS: Genome management system; HPO™: Human Phenotype Ontology™; LIMS™: Clarity laboratory information management system. Data types were as follows: *: HL7/FHIR; †: JSON; ‡: bcl; □: vcf.

### Supplementary Materials (Example 1)

Tables

TABLE 1
Duration and metrics for the major steps in the diagnosis of genetic diseases by genome sequencing using
rapid standard methods (Std.) and a rapid, autonomous platform (Auto.). Primary (1°) and secondary (2°) Analysis: conversion
of raw data from base call to FASTQ format, read alignment to the reference genomes and variant calling. Tertiary (3°)
Analysis Processing: Time to process variants and phenotypic features and make them available for manual interpretation in Opal ™
interpretation software (Fabric Genomics) or to display a provisional, automated diagnosis(es) in MOON ™ interpretation software
(Diploid). Dev. Delay: global developmental delay. PPHN: Persistent pulmonary hypertension of the newborn. HIE: Hypoxic ischemic
encephalopathy, n.a.: not applicable. *lncluded time to thaw a second set of NovaSeq ™ reagents. Included
10:20 hours of downtime, with manual restarting of the job, due to data center relocation. Patients 263, 6124
and 3003 were retrospectively analyzed by the autonomous system. Patient 263
was analyzed two times by the autonomous system. Patients 6194, 290, 352, 362, 412, and
7072 were prospectively analyzed by both autonomous and standard diagnostic methods.

| Use Type | Retrospective patients | | | | Prosepctive Patients | | | | | |
|---|---|---|---|---|---|---|---|---|---|---|
| Subject ID | 263 | | 6124 | 3003 | 6194 | | 290 | | 352 | |
| Age | 8 days | | 14 years | 1year | 5 days | | 3 days | | 7 weeks | |
| Sex | ♀ | | ♂ | ♀ | ♀ | | ♂ | | ♀ | |
| Abbreviated | Neonatal seizures | | Rhabdo- | Dystonia, | Hypoglycemia, | | Pulmonary | | Diabetic | |
| Presentation | | | myolysis | Dev. delay | seizures | | hemorrage, PPHN | | ketozcidosis | |
| Method | Auto. | Auto. | Auto. | Auto. | Auto. | Std. | Auto. | Std. | Auto. | Std. |
| Number of | 51 | | 115 | 148 | 14 | 2 | 257 | 4 | 103 | 4 |
| Phenotypic | | | | | | | | | | |
| Features | | | | | | | | | | |
| Molecular | Early Infantile | | Glycogen | Dopa- | None | None | None | None | Permanent | |
| Diagnosis | Epileptic | | Storage | Responsive | | | | | neonatal | |
| | Encephalopathy | | Disease | Dystonia | | | | | diabetes mellitus | |
| | 7 | | V | | | | | | | |
| Gene and | KCNQ2 | | PYGM | | n.a. | n.a. | n.a. | n.a. | INS c.26C > G | |
| Causative | c.727C > G | | c.2262delA | TH c.785C > G | | | | | | |
| Variant(s) | | | c.1726C > T | c.541C > T | | | | | | |
| Sample/Library | 3:20 | 2:55 | 2:24 | 2:22 | 2:10 | 23:54 | 2:12 | 22:05 | 2:13 | 15:42 |
| Prep (hours) | | | | | | | | | | |
| NovaSeq | | | | | | | | | | |
| Loading (hours) | 0:20 | 0:17 | 0:16 | 0:20 | 1:38* | 0:20 | 0:29 | 0:22 | 0:30 | 0:53 |
| 2 × 101 nt | | | | | | | | | | |
| Sequencing | 15:36 | 15:31 | 15:34 | 15:27 | 15:26 | 24:13 | 15:25 | 24:08 | 15:21 | 22:44 |
| (hours) | | | | | | | | | | |
| 1° & 2° Analysis | 1:03 | 1:02 | 0:59 | 0:59 | 1:07 | 3:05 | 1:00 | 1:57 | 1:01 | 2:30 |
| (hours) | | | | | | | | | | |
| 3° Analysis | | | | | | | | | | |
| Processing | 0:06 | 0:05 | 0:07 | 0:05 | 0:06 | 0:15 | 0:08 | 0:14 | 0:06 | 0:15 |
| (hours) | | | | | | | | | | |
| Total (hours) | 20:25 | 19:56 | 19:20 | 19:14 | 20:42* | 56:03 | 19:29 | 48:46 | 19:11 | 42:04 |

| Use Type | Prosepctive Patients | | | | | | | |
|---|---|---|---|---|---|---|---|---|
| Subject ID | 362 | | 374 | | 7052 | | 412 | |
| Age | 4 weeks | | 2 days | | 17 months | | 3 days | |
| Sex | ♂ | | ♂ | | ♂ | | ♂ | |
| Abbreviated | Neonatal | | HIE, | | Pseudomonal | | Neonatal | |
| Presentation | seizures | | anemia | | septic shock | | seizures | |
| Method | Auto. | Std.. | Auto. | Std. | Auto. | Std. | Auto. | Std. |
| Number of | 65 | 1 | 112 | 6 | 124 | 3 | 33 | 1 |
| Phenotypic | | | | | | | | |
| Features | | | | | | | | |

| Molecular | None | None | None | None | X-linked | Benign familial |
|---|---|---|---|---|---|---|
| Diagnosis | | | | | agamma- | neonatal seizures |
| | | | | | globulinemia 1 | 1 |

| Gene and | n.a. | n.a. | n.a. | n.a. | BTK c.974 + 2T > C | | KCNQ2 | |
|---|---|---|---|---|---|---|---|---|
| Causative | | | | | | | .1051C > G | |
| Variant(s) | | | | | | | | |
| Sample/Library | 2:31 | 18:30 | 3:30 | 10:10 | 4:30 | 12:10 | 3:05 | 23:50 |
| Prep (hours) | | | | | | | | |
| NovaSeq | | | | | | | | |
| Loading (hours) | 0:15 | 2:30 | 0:45 | 0:35 | 1:00 | 1:00 | 0:20 | 0:53 |
| 2 × 101 nt | | | | | | | | |
| Sequencing | 15:17 | 33:36 | 15:17 | 21:07 | 15:19 | 22:46 | 15:58 | 21:00 |
| (hours) | | | | | | | | |
| 1° & 2° Analysis | 1:02 | 2:30 | 1:02 | 2:30 | 1:09 | 2:25 | 1:24 | 2:24 |
| (hours) | | | | | | | | |
| 3° Analysis | | | | | | | | |
| Processing | 0:05 | 0:15 | 10:28^{†} | 0:16 | 0:06 | 0:16 | 0:06 | 0:16 |
| (hours) | | | | | | | | |
| Total (hours) | 19:10 | 57:21 | 31:02^{†} | 34:38 | 22:04 | 38:37 | 20:53 | 48:23 |

TABLE 2
Comparison of the analytic performance of standard and new library preparation, and standard and rapid genome sequencing
in retrospective samples. The standard library preparation and genome sequencing methods were TruSeq™ PCR-free library preparation and 2 ×
100 nt sequencing on a NovaSeq ™ 6000 with S2 flow cell, respectively. The new library preparation and genome sequencing methods were
Nextera Flex ™ library preparation and 2 × 100 nt sequencing on a NovaSeq ™ 6000 with SI flow cell, respectively. The “Median” column is
the median of runs R17AA978, R17AA978, R17AA059, and R17AA119. Controls 1 and 2 are mean values for five and fifty-two samples,
respectively. Analytic performance of variant calls was assessed in sample NA12878, with comparison to the NIST Genome-in-a-bottle results
(76). Note: The NA12878 control run with the SI flowcell and TruSeq ™ PCR free library (far right) was 2 × 151 nt.

| Run | | | | | | | | | |
|---|---|---|---|---|---|---|---|---|---|
| NovaSeq ™ 6000 | | | | | Median | NA12878 | Control 1 | Control 2 | NA12878 |
| Flowcell | R17AA978 | R17AA978 | R17AA059 | R17AA119 | S1 | | S2 | | |
| Library Preparation | S1 | | | | Nextera ™ Flex | | Nextera ™ | S2 | S1 |
| Method | Nextera ™ Flex | | | | 263 × 2, | | Flex | TruSeq ™ | PCR-free |
| Sample | 263 | 263 | 6124 | 3003 | 6124, 3003 | 1 sample | 5 samples | 52 samples | 1 sample |
| Raw Yield Per Flowcell (Gb) | 416 | 419 | 404 | 432 | 418 | 435 | 933 | 897 | 537 |
| % Reads Q > 30 | 92.00% | 92.07% | 92.11% | 94.84% | 92.09% | 90.69% | 91.50% | 91.70% | 91.96% |
| Trimmed Yield (Gb) | 153.9 | 158.9 | 165.0 | 160.7 | 159.8 | 148.9 | 183.3 | 152.8 | 164.5 |
| % Reads Mapped | 97.9% | 97.9% | 98.1% | 96.9% | 97.9% | 98.9% | 98.6% | 98.7% | 98.8% |
| % Duplicate Reads | 9.3% | 10.4% | 7.6% | 19.1% | 9.8% | 8.50% | 11.4% | 6.3% | 17.2% |
| Mean Insert Size (nt) | 386.0 | 348.0 | 336.0 | 274.0 | 342.0 | 345.1 | 315.1 | 423.4 | 514.6 |
| Average genome coverage | 42.0 | 43.0 | 44.4 | 39.0 | 42.5 | 47.5 | 49.4 | 43.6 | 32.9 |
| % OMIM genes with 100% | 96.0% | 95.7% | 94.9% | 65.1% | 95.3% | 95.8% | 96.8% | 97.7% | 98.00% |
| coverage at > 10X | | | | | | | | | |
| Variants | 4,910,055 | 4,915,843 | 4,847,506 | 4,655,831 | 4,878,781 | 4,733,000 | 4,976,974 | 4,922,188 | 4,747,231 |
| Variants passing QC | 96.0% | 96.1% | 96.6% | 96.8% | 96.3% | 96.8% | 98.1% | 98.4% | 98.5% |
| CD Variants | 0.53% | 0.53% | 0.55% | 0.54% | 0.53% | 0.58% | 0.53% | 0.53% | 0.58% |
| Indels | 17.8% | 17.9% | 18.0% | 17.5% | 17.8% | 17.5% | 18.6% | 18.8% | 19.4% |
| CD Homozygous/ | 0.59 | 0.59 | 0.57 | 0.60 | 0.59 | 0.60 | 0.56 | 0.59 | 0.60 |
| Heterozygous Variant | | | | | | | | | |
| Ratio | | | | | | | | | |
| Ti/Tv ratio | 2.02 | 2.02 | 2.02 | 2.03 | 2.02 | 2.02 | 2.02 | 2.02 | 2.01 |
| CD Ti/Tv ratio | 2.85 | 2.87 | 2.88 | 2.94 | 2.88 | 2.81 | 2.85 | 2.85 | 2.82 |
| Analytic Performance | | | | | | | | | |
| PPV (SNV) | n.a. | n.a. | n.a. | n.a. | n.a. | 99.8% | 99.8% | 99.9% | 99.9% |
| PPV (indels) | n.a. | n.a. | n.a. | n.a. | n.a. | 99.0% | 97.0% | 99.3% | 99.7% |
| Sensitivity (SNV) | n.a. | n.a. | n.a. | n.a. | n.a. | 99.7% | 99.6% | 99.7% | 99.8% |
| Sensitivity (indels) | n.a. | n.a. | n.a. | n.a. | n.a. | .95.5% | 96.3% | 99.0% | 99.4% |

Abbreviations: nt: Nucleotides; FC: flowcell; Gb: gigabase; Q: Quality score; OMIM ™: Online Mendelian Inheritance ™ in Man; QC: Quality Control; CD: Coding Domain; Ti/Tv ratio: ratio of the number of nucleotide transitions to the number of nucleotide transversions; PPV: Positive predictive value; SNV: single nucleotide variants; indels: nucleotide insertion-deletion variants.

TABLE 3
Comparison of the analytic performance of standard and new library preparation and genome sequencing methods in seven matched
prospective samples. The standard library preparation and genome sequencing methods were TruSeq ™ PCR-free library preparation
and NovaSeq ™ 6000 with S2 flow cell, respectively, with the exception of subjects 7052 and 412, where the library preparation was
done with the KAPA Hyper ™ kit. The new library preparation and genome sequencing methods were Nextera™ Flex library
preparation and NovaSeq ™ 6000 with S1 flow cell, respectively.

| Run | R18AA202 | Std. | R18AA218 | Std. | R18AA922 | Std | R18AB113 | Std |
|---|---|---|---|---|---|---|---|---|
| Subject | 6194 (Prospective) | | 290 (Prospective) | | 352 (Prospective) | | 362 (Prospective) | |
| Library Prep Method | Nextera | TruSeq | Nextera | TruSeq | Nextera | TruSeq | Nextera | TruSeq |
| Flow cell | S1 | S2 | S1 | S2 | S1 | S2 | S1 | S2 |
| Raw Yield Per Flow cell | 389.9 | 945.4 | 381.8 | 946 | 365.3 | 869.9 | 398.3 | 440.7 |
| (Gb) | | | | | | | | |
| Reads Q >= 30 | 90.90% | 93.70% | 91.30% | 93.10% | 89.80% | 90.70% | 92.20% | 90.00% |
| % Cluster passing filter, | 69.8/82.9 | 82.1/82.0 | 73.9/75.6 | 82.2/82.0 | 73.8/69.3 | 75.5/75.5 | 78.9/77.1 | 36.7/39.9 |
| L1/L2 | | | | | | | | |
| % Error rate (ΦX174), | 0.19/0.42 | 0.27/0.47 | 0.25/0.65 | 0.27/0.37 | 0.25/0.45 | 0.31/0.37 | 0.20/0.36 | 0.33/0.41 |
| R1/R2 | | | | | | | | |
| Trimmed Yield (Gb) | 174.1 | 172.3 | 168.6 | 218.2 | 141 | 144.2 | 164.3 | 148.4 |
| Reads Mapped | 97.70% | 98.60% | 97.30% | 98.30% | 97.20% | 98.60% | 97.40% | 98.50% |
| Duplicate Reads | 11.50% | 6.50% | 11.60% | 7.30% | 8.90% | 9.20% | 9.90% | 3.90% |
| Mean Insert Size | 361.2 | 405.8 | 223.7 | 430 | 373.4 | 419.8 | 369 | 410 |
| (nt) | | | | | | | | |
| Average genome coverage | 44.8 | 48.4 | 54 | 60.4 | 39.1 | 39.3 | 43.1 | 42.8 |
| % OMIM genes w. > 10X × | 95.80% | 97.90% | 93.30% | 98.20% | 95.80% | 97.80% | 95.70% | 96.60% |
| 100% nt | | | | | | | | |
| Variants | 4,687,590 | 4,881,456 | 4,776,648 | 5,016,422 | 4,765,467 | 4,934,554 | 4,719,091 | 4,917,044 |
| Variants passing QC | 96.90% | 98.30% | 97.00% | 98.20% | 97.00% | 98.60% | 97.00% | 98.20% |
| CD Variants | 0.57% | 0.52% | 0.57% | 0.53% | 0.54% | 0.56% | 0.55% | 0.54% |
| Indels | 18.20% | 18.90% | 18.00% | 18.90% | 18.00% | 18.60% | 17.70% | 18.50% |
| Ti/Tv ratio | 2.02 | 2.02 | 2.03 | 2.03 | 2.02 | 2.03 | 2.02 | 2.01 |

| Run | R18AB229 | Std | R18AB352 | Std | R18AB672 | Std |
|---|---|---|---|---|---|---|
| Subject | 374 (Prospective) | | 7052 (Prospective) | | 412 (Prospective) | |
| Library Prep Method | Nextera | KAPA | Nextera | KAPA | Nextera | KAPA |
| | | Hyper | | Hyper | | Hyper |
| Flow cell | S1 | S2 | S1 | S2 | S1 | S2 |
| Raw Yield Per Flow cell | 420.8 | 899.1 | 383.4 | 860.2 | 422.1 | 908.2 |
| (Gb) | | | | | | |
| Reads Q >= 30 | 93.30% | 91.60% | 90.10% | 90.10% | 92.90% | 91.60% |
| % Cluster passing filter, | 83.0/81.8 | 78.3/77.8 | 75.49/74.7 | 75.2/74.1 | 83.1/82.3 | 78.9/78.8 |
| L1/L2 | | | | | | |
| % Error rate (ΦX174), | 0.20/0.40 | 0.25/0.35 | 0.26/0.50 | 0.31/0.36 | 0.22/0.32 | 0.28/0.29 |
| R1/R2 | | | | | | |
| Trimmed Yield (Gb) | 185.5 | 267.8 | 156.4 | 138 | 183.4 | 203 |
| Reads Mapped | 98.00% | 98.50% | 97.30% | 98.30% | 98.60% | 98.60% |
| Duplicate Reads | 11.70% | 14.60% | 8.30% | 9.40% | 14.00% | 13.40% |
| Mean Insert Size | 266.9 | 423.8 | 371.4 | 428.4 | 338.1 | 416.2 |
| (nt) | | | | | | |
| Average genome coverage | 48 | 68.4 | 41.6 | 37.3 | 47.6 | 50.9 |
| % OMIM genes w. > 10X × | 96.00% | 98.40% | 95.20% | 97.80% | 96.90% | 98.20% |
| 100% nt | | | | | | |
| Variants | 4,758,713 | 5,001,708 | 4,821,433 | 4,981,748 | 4,958,194 | 4,965,915 |
| Variants passing QC | 98.10% | 98.00% | 98.10% | 98.60% | 98.10% | 98.20% |
| CD Variants | 0.55% | 0.53% | 0.56% | 0.53% | 0.56% | 0.53% |
| Indels | 19.60% | 18.80% | 17.60% | 18.50% | 18.70% | 18.90% |
| Ti/Tv ratio | 2.01 | 2.01 | 2.03 | 2.02 | 2.01 | 2.02 |

Abbreviations: L: lane R: read; nt: Nucleotides; Gb: gigabase; Q: Quality score; OMIM ™: Online Mendelian Inheritance in Man ™; QC:Quality Control; CD: Coding Domain; Ti/Tv ratio: ratio of the number of nucleotide transitions to the number of nucleotide transversions.

TABLE 4
Characteristics of sixteen children with genetic diseases used to train CNLP.

| | | | | | | | de | |
|---|---|---|---|---|---|---|---|---|
| | | | | | | | novo | |
| | S, | rWES | | | | | or | |
| | D, | or | | | OMIM | Inherit- | inher- | |
| Family | T, | rWGS | Disease | Affected Gene | ID | ance | ited | Variant 1 (V1) |
| 6007 | T | rWGS | EIEE9 | PCDH19 | 300088 | AD | DN | Xq22del |
| 6008 | S | rWGS | Glioblastoma | BRCA1 | 604370 | AD | n.d. | c.5159G > A, p.Arg1720Gln |
| | | | | | | | | c.3096_3100delCAAAG; |
| 6012 | S | rWGS | Coffin-Siris syndrome 1 | ARIDIB | 135900 | AD | DN | p.LyslO33ArgfsTer32 |
| 6014 | S | rWGS | Nemaline myopathy 2 | NEB | 256030 | AR | n.d. | c.19262 + 1G > A |
| 6024 | T | rWGS | Hypophosphatemic rickets, X-linked | PHEX | 307800 | XLD | I | c.1604C > T,p.Thr535Met |
| | | | dominant | | | | | |
| 6026 | T | rWGS | Alagille syndrome 1 | 20p12.2del | 118450 | AD | DN | Chr20:10471400-13459331del |
| 6030 | T | rWGS | Neurofibromatosis 1; Left | NF1 & MYBPC3 | 162200, | AD, | DN, | c.5118delT, |
| | | | ventricular noncompaction 10 | | 615396 | AD | I | p.Val1707PhefsTer |
| 6031 | T | rWGS | Catecholaminergic | RYR2 | 604772 | AD | DN | c.1646OT; p.Ala549Val |
| | | | polymorphicVentricular tachycardia | | | | | |
| 6037 | T | rWGS | 1 | none | none | n.a. | n.a. | n.a. |
| | | | Neonatal cholestasis; Extrahepatic | | | | | |
| 6041 | T | rWGS | biliary atresia | KCNQ2 | 613720 | AD | DN | c.875T > C; p.Leu292Pro |
| | | | EIEE7 | | | | | |
| 6044 | S | rWGS | Pleuropulmonary blastoma | DICER | 601200 | AD | n.d. | c.2771T > G; p.Leu924* |
| 6045 | S | rWGS | Medulloblastoma | none | none | n.a. | n.a. | n.a. |
| 6051 | S | rWGS | Glioma | none | none | n.a. | n.a. | n.a. |
| 6052 | T | rWGS | MECRCN | TANGO2 | 616878 | AR | I | c.605 + 1G > A |
| 6066 | D | rWGS | Neonatal cholestasis; Cleft lip and | none | none | n.a. | n.a. | n.a. |
| | | | palate | none | | | | |
| 6117 | D | rWGS | | Neonatal cholestasis | none | n.a. | n.a. | n.a. |
| | | | | | | Age at | | |
| | | | | V1 | V2 | enroll- | | |
| | | | | P/L | P/L | ment | | |
| | | Family | Variant 2 (V2) | P | P | (days) | Sex | Consanguinity |
| | | 6007 | | | | 423 | F | No |
| | | 6008 | | | | 4563 | F | No |
| | | 6012 | | | | 231 | F | |
| | | 6014 | c.2416-1G > C | | | 35 | M | No |
| | | 6024 | | | | 137 | M | No |
| | | 6026 | | | | 80 | M | U |
| | | 6030 | c.3184delG p.Val1062LeufsTer13 | LP | LP | 227 | M | No |
| | | 6031 | | | | 6087 | F | No |
| | | 6037 | | | | 60 | M | U |
| | | 6041 | | | | 2 | F | No |
| | | 6044 | | | | 564 | M | U |
| | | 6045 | | | | 5475 | M | U |
| | | 6051 | | | | 2555 | M | U |
| | | 6052 | 33 kb del TANGO2 exons 3-9 | | | 898 | F | U |
| | | 6066 | | | | 60 | F | U |
| | | 6117 | | | | 60 | F | U |

Abbreviations:
EIEE: Early Infantile Epileptic Encephalopathy;
AD: Autosomal Dominant;
DN: de novo;
P: Pathogenic;
LP: Likely Pathogenic;
M: Male;
F: Female;
S: Singleton;
D: Duo;
T: Trio;
I: Inherited;
XLD: X-linked dominant;
MECRN: Metabolicencephalomyopathic crises, recurrent, with rhabdomyolysis, cardiac arrhythmias, and neurodegeneration;
U: undetermined;
OMIM: OnlineMendelian Inheritance in Man.

TABLE 5
Precision and recall of phenotypic features extracted by CNLP from EHRs in ten children with genetic diseases.
Precision = tp/tp + fp. Recall = tp/tp + f.

| | | | | | | | de | | |
|---|---|---|---|---|---|---|---|---|---|
| | | | | | | | novo | | |
| | S | rWES | | | | | or | | |
| | or | or | | Affected | OMIM | Inher- | inher | | |
| Family | T | rWGS | Disease | Gene | ID | itance | -ited | Variant 1 (V1) | Variant 2 (V2) |
| 201 | T | rWES | Prader Willi | 15q11- | 176270 | AD | DN | Chr15:23684685- | |
| | | | Syndrome | q13 del | | | | 26108259del | |
| 205 | T | rWGS | Dursun Syndrome | G6CP3 | 612541 | AR | I | c.207dupC, | c.199)_218 + 1delCTCAACC |
| | | | | | | | | p.IIe70HisfsTer17 | TCATCTTCAAGTGG |
| 213 | S | rWGS | Visceral Heterotaxy 5 | NODAL | 270100 | AD | I | c.778G > A, p.Gly260Arg | |
| 233 | T | rWGS | Tuberous Sclerosis 1 | TSC1 | 191100 | AD | DN | c.1498C > T, | |
| | | | | | | | | p.Arg500Ter | |
| 243 | T | rWGS | Pyridoxine | ALDH7A1 | 266100 | AR | I | c.328C > T, | c.1279G > C, |
| | | | dependent seizures | | | | | p.Arg110Ter | p.Glu427Gln |
| 6094 | T | rWGS | Argininosuccinic | ASL | 207900 | AR | I | c.706C > T, | c.706C > T |
| | | | Aciduria | | | | | p.Arg236Trp | p.Arg236Trp |
| 6098 | T | rWGS | Gaucher disease | GBA | 230800 | AR | I | c.1503C > G, | c.1448T > C, |
| | | | | | | | | p.Asn501Lys | p.Leu483Pro |
| 6108 | T | rWGS | Tuberous Sclerosis 2 | TSC2 | 613254 | | DN | c.935_936delTC, | |
| | | | | | | | | p.Leu312GlnfsTer25 | |
| 7003 | T | rWGS | EIEE6 | SCN1A | 607208 | | DN | c.5555T > C, | |
| | | | | | | | | p.Met1852Thr | |
| 7004 | T | rWGS | Hypertrophic | MYH7 | 192600 | | I | c.746G > A, | |
| | | | cardiomyopathy type | | | | | p.Arg249Gln | |
| | | | 1 | | | | | | |
| Mean | | | | | | | | | |
| | Standard Deviation | | | | | | | | |
| | | | Age at | | | | | | |
| | V1 | V2 | enroll- | | | | | | OMIM CF |
| | P/ | P/ | ment | | Consan- | CNLP | CNLP | CNLP | detected |
| Family | LP | LP | (days) | Sex | guinity | Features | Precision | Recall | by CNLP |
| 201 | | | 3 | ♀ | U | 26 | 0.88 | n.d. | 3% |
| 205 | P | P | 2 | ♂ | No | 96 | 0.80 | n.d. | 15% |
| 213 | | | 3 | ♂ | U | 95 | 0.67 | 0.91 | 56% |
| 233 | | | 3 | ♀ | No | 158 | 0.51 | 0.91 | 14% |
| 243 | | | 7 | ♂ | No | 85 | 0.82 | 0.93 | 21% |
| 6094 | P | P | 7 | ♀ | Yes | 90 | 0.83 | | 11% |
| 6098 | | | 214 | ♀ | No | 96 | 0.9 | | 21% |
| 6108 | | | 3 | ♂ | No | 83 | 0.76 | | 5% |
| 7003 | | | 424 | ♂ | U | 44 | 0.84 | 0.93 | 25% |
| 7004 | | | 5171 | ♂ | U | 71 | 0.94 | 0.96 | 44% |
| Mean | | | | | | 86.7 | 0.80 | 0.93 | 22% |

| | | | | |
|---|---|---|---|---|
| Standard Deviation | 32.8 | 0.13 | 0.02 | 0.17 |

Abbreviations:
E1EE: Early Infantile Epileptic Encephalopathy;
AD: Autosomal Dominant;
AR: Autosomal Recessive;
DN: de novo;
P: Pathogenic;
LP: Likely Pathogenic;
S: Singleton;
T: Trio;
I: Inherited;
U: undetermined;
OMIM: Online Mendelian Inheritance in Man;
CF: Clinical Feature.

TABLE 6
Number of structural variants shortlisted by MOON ™ and
rank of the causal variant in MOON ™ in 11 children
with genetic diseases. All samples were run as singletons.

| | | | # SV | Causal |
|---|---|---|---|---|
| | | # SV | shortlisted | SV rank |
| Family | rWES/rWGS | calls in gVCF | by MOON | in MOON |
| 201 | rWES | 6 | 2 | 1 |
| 259 | rWES | 16 | 9 | 1 |
| 286 | rWES | 7 | 3 | 1 |
| 319 | rWES | 12 | 4 | 1 |
| 217 | rWGS | 21 | 8 | 1 |
| 223 | rWGS | 16 | 9 | 5 |
| 302 | rWGS | 22 | 17 | 13 |
| 6140 | rWGS | 11 | 8 | 1 |
| 6146 | rWGS | 23 | 15 | 9 |
| 6164 | rWGS | 25 | 15 | 12 |
| 7023 | rWGS | 17 | 12 | 12 |
| Mean, rWES | | 10.3 | 4.5 | Median rWGS, |
| Mean, rWGS | | 19.3 | 12.0 | rWES 1.0 |

Abbreviations: gVCF: Genomic variant call file; rWES: rapid whole exome sequencing; rWGS: rapid whole genome sequencing; SV: structural variant.

TABLE 7
Summary statistics of provisional diagnoses reported for rapid clinical
genome sequencing. Total probands refers to children tested.

| | | Mean Time to |
|---|---|---|
| | | Provisional Report |
| | | (Sample Accession to |
| | | Preliminary Results |
| Total Probands | Provisional Reports Returned | Communicated), Days |
| 684 | 114 (16.7%) | 3.6 |

### Example 2

### Automated System and Method for Population-Scale Diagnosis and Acute Management Guidance for Genetic Diseases

In this example, a system of automated diagnosis and acute management guidance for genetic diseases in critically ill children in 13.5 hours is described that will facilitate population-scale implementation.

Experimental Materials and Methods

Study Design.

This study reports results from human subject research approved by the institutional review board at Rady Children's Hospital, San Diego, and the University of California—San Diego, which were performed in accordance with the Declaration of Helsinki. Informed, written consent was obtained from at least one parent or guardian of the participating infants. Families were not compensated for participation. Datasets were obtained from four retrospectively studied infants (age less than one year, two male and two female) and three prospectively studied male neonates (aged less than 28 days) to test the analytic, diagnostic, and clinical management performance of the 13.5-hour method. Ten cases (six male and four female, seven neonates, two older infants, and one 14-year old) used to verify the analytic performance of the clinical natural language processing were identified from research study populations. Four retrospective cases were identified from recent clinical operations at Rady Children's Institute for Genomic Medicine (RCIGM). All had received recent diagnoses by rWGS®, performed in the RCIGM CLIA/CAP laboratory, and blood sample retains were used for comparative re-analysis by the 13.5-hour method. Three prospective cases were also ascertained from RCIGM clinical operations. Prospective cases received both standard rWGS® performed according to CLIA/CAP standards and the prototypic 13.5-hour method concomitantly. Provisional results from the prototypic 13.5-hour method were returned to the attending neonatologist before confirmation by the standard method in accordance with a determination of “nonsignificant risk” by the FDA in response to an Investigational Device Exemption pre-submission enquiry for the antecedent study in April 2014. This study also reports results of a quality improvement project for diagnostic rWGS® performed at Rady Children's Institute for Genomic Medicine (RCIGM) laboratory in conformity with the College of American Pathologists (CAP) and Clinical Laboratory Improvement Amendments (CLIA) standards.

Natural Language Processing and Phenotype Extraction.

Human Phenotype Ontology™ (HPO™, github.com/obophenotype/human-phenotype-ontology/blob/master/src/ontology/reports/hpodiff_hp_2021-06-13_to_hp_2021-08-02.xlsx) terms for cases with a Rady Children's Hospital Epic EHR were automatically extracted in four steps by natural language processing (NLP) of text fields: (1) Clinical records were exported from the Epic™ EHR data warehouse, transformed into a compatible format (JSON), and loaded into CLiX ENRICH™ v.6.7 (CliniThink™ Ltd.). (2) A semi-automated query map was created, with HPO terms (and their synonyms) as the input and CLiX™ queries as the output. The HPO terms were passed through the CLiX™ encoding engine, resulting in creation of CLiX™ post-coordinated SNOMED CT™ (confluence.ihtsdotools.org/display/RMT/SNOMED+CT+January+2022+International+Edition+−+SNOMED+International+Release+notes) expressions for each recognized HPO term or synonym. Where matches were not exact, manual review was used to validate the generated CLiX™ queries. Where there was no match or incorrect matches, new content was added to the Clinithink™ SNOMED CT™ extension and terminology files to ensure appropriate matches between phenotypes in HPO and those in SNOMED CT™. This was an iterative process that resulted in a CLiX™ query set that covered 60% (7706) of 12,786 HPO terms. (3) EHR documents containing unstructured data were passed through the NLP™ engine. The NLP™ processing engine read the unstructured text and encoded it in structured format as post-coordinated SNOMED CT™ expressions. These encoded data were then interrogated by the CLiX™ query technology (abstraction). To trigger an HPO query, the encoded data had to contain either an exact match or one of its logical descendants (exploiting the parent-child hierarchy of the SNOMED CT™ ontology), resulting in a list of HPO terms for each patient. EHR data for cases from partner hospitals was imported as machine-readable .pdf files to CLX™ ENRICH™ v.6.7. In cases with more than one .pdf file, they were combined into a .zip file for upload to CLiX™ ENRICH™. The NLP™ engine read the unstructured text and encoded it as HPO terms, resulting in a list of observed terms for each patient.⁵⁵ The analytic performance of NLP by CLiX™ ENRICH™ v.6.7 and v.6.5 was compared with manual chart review by two physician experts for ten test cases.

Rapid Diagnostic Whole Genome Sequencing.

The standard clinical rWGS® methods were DNA isolation from EDTA blood samples with the EZ1™ DSP DNA Blood Kit (Qiagen, Cat. No. 62124), followed by library preparation with the polymerase chain reaction (PCR)-free KAPA HyperPrep™ kit (Roche, Cat. No. KK8505), and 2×101 nucleotide (nt) sequencing on NovaSeq™ 6000 instruments (Illumina, Cat. No. 20013850) with 51 flowcells, v.1 reagents, and standard recipe (Illumina, Cat. No. 20028319). The 19.5-hour rWGS® methods were library preparation from EDTA blood samples with Nextera™ DNA Flex Library Prep kits (Illumina, Cat. No. 20018705) and five cycles of PCR, 2×101 nt sequencing without indexing on NovaSeq™ 6000 instruments with 51 flowcells, v.1.0 reagents, and a custom recipe with accelerated cycle time (Illumina, Cat. No. 20012864), and sequence alignment and nucleotide variant detection with the DRAGEN™ Platform (v.2.5.1, Illumina, Cat. No. 20060401).

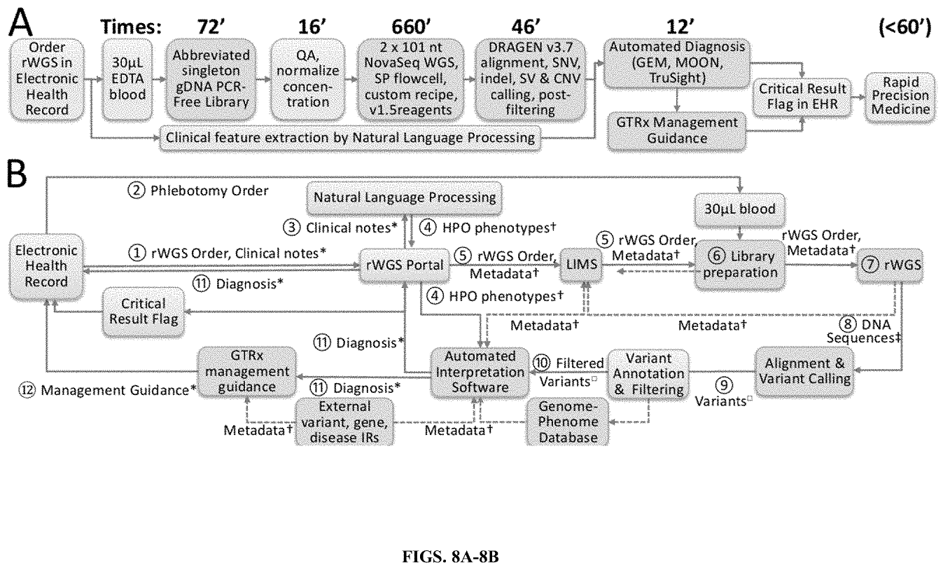

For 13.5-hour rWGS®, sequencing libraries were prepared directly from EDTA blood samples or five 3 mm² punches from a Nucleic Card Matrix dried blood spot (ThermoFisher, Cat. No. 4473977), without intermediate DNA purification, using magnetic bead-linked transposomes (DNA PCR-free Prep kit, Tagmentation, Illumina, Cat. No. 20041795). The length of each incubation step was maximally reduced from those in the manufacturer's protocol (FIG. 8). The shorter incubations normalized library output, which enabled simpler, faster measurement of library concentration with a KAPA™ Library Quantification Kit (Roche, Cat. No. 07960140001). 2×101 cycle sequencing-by-synthesis was performed on NovaSeq™ 6000 instruments (Illumina, Cat. No. 20013850) with a custom instrument run recipe with maximally reduced cycle time consistent with retention of sequence quality. Sequencing used SP flowcells and version 1.5 reagents (Illumina, Cat. No. 20040719), which were more cost effective and delivered better sequence quality than v.1.0 reagents. Sequences were aligned to human genome assembly GRCh37 (hg19), and variants identified and genotyped with the DRAGEN™ platform v.3.7.5 (Illumina). Automated variant interpretation was performed in parallel using MOON™ (InVitae), GEM™ (Fabric Genomics), and the Illumina TruSight™ Software Suite (TSS™, Illumina).^{16,39} Inputs were the variant call file (vcf), list of observed HPO terms, and patient metadata (coded identifier, name, EHR number, ordering physician, date of birth, location, relationship to proband). All three software platforms (MOON™, GEM™, and TSS™) generated a list of potential provisional diagnoses by sequentially filtering and ranking variants using decision trees, Bayesian models, neural networks, and natural language processing. The three software platforms ranked variants according to phenotypic match, pathogenicity, and rarity (Table 12). For generalizable, high throughput clinical use, each of these components was integrated with a custom laboratory information management system (LIMS™, L7 Inc.) and custom analysis pipeline (Axolotl™ v.5.0, Rady Children's Institute for Genomic Medicine) that automated data transfers between steps.

Measurement of Analytic Performance of rWGS®.

The analytic performance of the new rWGS® methods was compared with prior clinical rWGS® methods in two reference DNA samples (NA12878, catalog.coriell.org/O/Sections/Search/Sample_Detail.aspx?Ref=NA12878, and NA24385, catalog.coriell.org/O/Sections/Search/Sample_Detail.aspx?Ref=NA24385&Product=DNA) using NIST gold standard variant sets for SNVs and indels (NISTv4.1, ftp-trace.ncbi.nlm.nih.gov/giab/ftp/release/), and SVs and CNVs (NISTv0.6, ftp-trace.ncbi.nlm.nih.gov/ReferenceSamples/giab/data/AshkenazimTrio/analysis/NIST_SVs_Integration_v0.6/) and Witty.er v0.3.4 (github.com/Illumina/witty.er/releases).

Gene and Intervention Curation.

358 genes associated with 563 critical, childhood-onset illness with effective treatments were identified by literature review, subspecialist nomination and rapid precision medicine experience (data not shown). Automated scripts were written to collect information about the gene, inheritance pattern, natural history and interventions from publicly available information resources. Gene to disease mapping was done using OMIM™ (omim.org/) and Orphanet (orpha.net/consor/cgi-bin/Disease.php?lng=EN) mappings. Resources included OMIM™, Orphanet™, Clinical Trials™ (clinicaltrials.gov/ct2/home), ClinVar™ (ncbi.nlm.nih.gov/clinvar/), clinical trial registries including the Cochrane database (cochranelibrary.com/central/about-central), DrugBank™ v5.0 (go.drugbank.com/releases/latest), Gene™ (ncbi.nlm.nih.gov/gene), Genetic and Rare Disease Information Center™ (GARD™) (rarediseases.info.nih.gov/diseases), GeneReviews™ (ncbi.nlm.nih.gov/books/NBK1116/), Inxight:Drugs™ (drugs.ncats.io/substances), GHR™ (medlineplus.gov/genetics/gene/ghr/), MedGen™ (ncbi.nlm.nih.gov/medgen/), Medscape™ (reference.medscape.com/), NORD™ (rarediseases.org/for-patients-and-families/information-resources/rare-disease-information/), and PubMed™ (pubmed.ncbi.nlm.nih.gov/). Scripts were also written to identify published literature relating to each condition and identify pertinent treatments (Genomenon™ Inc. Rancho Biosciences™, Epam™). Publications were included if they mentioned the condition, the specific variant identified, and a clinical intervention used to treat the condition. Intervention lists for each gene-condition association were curated manually for relevance and specificity to the intensive care setting.

Expert Review Panel.

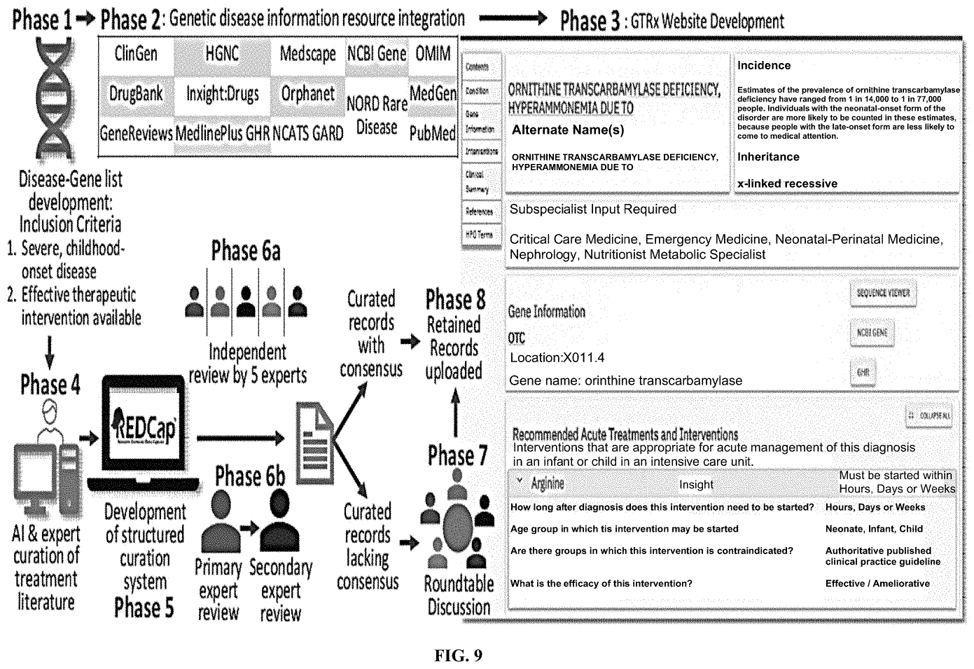

The list of interventions for each gene-condition association was adjudicated by a group of expert reviewers. Reviewers were experts in the fields of clinical and biochemical genetics. Five reviewers in total were recruited for the first stage of interface development. Software for intervention review was developed using the RedCap™ interface (RedCap™, redcap.radygenomiclab.com/redcap_v10.6.3/DataEntry/record_status_dashboard.php?pid=62), and reviewers were able to login via a web portal in order to review genes that had been curated by a combination of AI and manual curation. Expert consensus on curated interventions was required for the inclusion on the final user interface, as illustrated in FIG. 9. In Phase 1, reviewers were provided with a prototype set of 10 genes in order to test the reviewer interface, after which a concordance analysis was performed and the RedCap™ interface was extensively revised in response to reviewer feedback. The reviewers then reviewed the same 10 gene set again, with an additional 5 genes associated with pre-selected retrospective cases. Reviewers chose whether to retain or delete previously curated interventions, and indicated in what age group the intervention may be initiated, in what time frame after diagnosis the intervention would optimally be initiated, contraindications, efficacy, and level of evidence available in support of the intervention (Box 1). A set of core inclusion and exclusion criteria for interventions was drafted and revised by the group, as detailed in the Supplementary Materials. After initial review of the 15 gene pilot set, the interventions on which consensus was not reached were discussed in roundtable discussion. In Phase 2, reviewers were split into pairs, and each gene had one reviewer perform a primary review, and a second reviewer perform a secondary review (FIG. 9). Any disagreements between the primary and secondary expert review were again discussed in the roundtable meeting with all reviewers, and only interventions that reached full consensus were included. The final list of interventions was collated after full consensus had been reached between all five reviewers. As a final quality control and assurance step, an independent expert performed a final quality check for each gene before moving it to the user interface pipeline.

Box 1. Minimal, structured data elements required for FAIR-
compliant systematic literary reviews to create a virtual
acute management support system for clinicians.

Disease, gene, incidence, inheritance mode(s)
Appropriate subspecialist consultant(s)
Clinical summary/natural history of disease
Set of appropriate acute treatments:
Drug(s)
Device(s)
Diet(s)
Surgical intervention(s)
For each treatment:
Efficacy in this disease
Curative
Effective/Ameliorative
Still in Trials
Contraindicated
Evidence supporting efficacy in this disease
Authoritative published guidelines
Cohort study or studies
Case reports
Optimal timeframe to initiate after disease diagnosis
Hours
Days/Weeks
Years
Appropriate age group(s) in this disease
Neonates
Infants
Children
Contraindicated groups in this disease
Banner warning (if any)

User Interface Development and Integration into Automated Pipeline.

A web resource integrated the GTRx℠ information resources and the adjudicated interventions (gtrx.rbsapp.net/). The user interface for GTRx℠ was developed in partnership with Rancho Biosciences™. Automated scripts integrated the electronic acute disease management support system into MOON™ (Diploid), GEM℠ (Fabric Genomics), and the Illumina TruSight™ Software Suite (Illumina). This provided an automated link to treatment guidance once a provisional genetic diagnosis was reached by the variant curation tool. The provisional management plan automatically generated by GTRx℠ for each of the four retrospective cases were checked by a lab director and a clinician for accuracy.

Data Availability.

Source data are provided with this paper. The processed patient data generated in this study have been deposited in the Longitudinal Pediatric Data Resource™ (LPDR™) under accession code nbs000003.v1.p at nbstrn.org/. LPDR™ data are available under restricted access since it is pseudonymized human subjects data that is subject to privacy and confidentiality issues, the terms of informed written consent documents, and state and federal laws. Qualified newborn screening researchers can obtain access by registration at nbstrn.org/login?token-expired=true&rel=/tools/lpdr. The raw patient data are protected and not available due to data privacy and confidentiality laws. Anonymized and pseudonymized patient data generated in this study, subject to the terms of informed written consent documents, and state and federal laws, are provided in the Supplementary Information/Source Data file. Non-human subjects data generated in this study are provided in the Supplementary Information/Source Data file. NIST data used in this study are available at ftp-trace.ncbi.nlm.nih.gov/giab/ftp/release/, and ftp-trace.ncbi.nlm.nih.gov/ReferenceSamples/giab/data/AshkenazimTrio/analysis/NIST_SVs_Integration_v0.6/.

Code Availability.

Witty.er is available at github.com/Illumina/witty.er. InterVar™ is available at github.com/WGLab/InterVar. GTRx℠ is available at gtrx.radygenomiclab.com/. CLIXEnrich™ is available from CliniThink™. Moon™ is available from Invitae or Diploid. The DRAGEN™ Platform and the Illumina TruSight™ Software Suite are available from Illumina. OPAL™ and GEMS™ are available from Fabric Genomics. The RCIGM portal, Axolotl™ pipeline, and L7 LIMS™ are available from https://github.com/rao-madhavrao-rcigm/gtrx. The GTRx℠ REDCap™ instance are available from github.com/rao-madhavrao-rcigm/gtrx.

Results

13.5-Hour Genome Sequencing.

Genetic disease diagnosis by rWGS® in 19.5 hours is previously described. However, clinical usefulness was limited by lack of scalability and insensitivity for copy number variants (CNVs) or structural variants (SVs), which underpin 20% of genetic diagnoses in children in ICUs. Inclusive of CNV and SV detection, turnaround time was >30 hours, which was insufficient for the most rapidly progressive childhood genetic diseases, such as neonatal encephalopathies. rWGS® was re-engineered to improve scalability, turnaround time, analytic performance for CNVs and SVs, and generalization to other healthcare systems (FIG. 8).

First, ordering of rWGS® was simplified. Orders are placed directly through the Epic EHR (FIG. 8). The test order and patient metadata is transferred from the EHR to a custom ordering portal. Second, a simpler, faster method of sequencing library preparation was developed that retained the capability to identify CNVs and SVs, using magnetic bead-linked transposomes (DNA polymerase chain reaction-free kit, Illumina). Incubation steps were maximally reduced from those in the manufacturer's protocol (FIG. 8). Resultant library preparation took an average of 45 minutes from purified genomic DNA, and 72 minutes from blood (Table 8). Thirdly, much faster 2×101 cycle sequencing-by-synthesis was developed on NovaSeq™ 6000 instruments (lllumina, average 11 hours 12 minutes). This employed a custom instrument run recipe with maximally reduced cycle time, and SP flowcells, which were imaged only on one surface of each of two lanes. Fourthly, a faster method for sequence alignment and variant calling (average 34 minutes for 120 GB of singleton genome sequence) was developed that also had greatly improved analytic performance for SVs and CNVs (Dynamic Read Analysis for GENomics, DRAGEN™ v.3.7, Illumina). Finally, for generalizable, scalable clinical use, each of these components (sample accessioning, library preparation, library quality assessment, sequencing and variant calling) was integrated with a custom laboratory information management system and custom analysis pipeline (Enterprise Science Platform™, L7 Informatics) that automated data transfers between steps.

The analytic performance and reproducibility of the combined method was evaluated in reference DNA samples in which benchmark variant sets have been established by the National Institute of Standards and Technology (NIST). The average time from DNA sample to completion of variant calling was 12 hours and 42 minutes, 35% less than the previous minimum (Table 8). The analytic performance for single nucleotide variants (SNVs) and insertion-deletion oligonucleotide variants (indels) was also improved, with precision and recall values>99.4% (Table 9).

The analytic performance of DRAGEN™ v.3.7 for structural variants (SVs, size>50 nt) and CNVs (size>10 kb) was compared with the widely used methods Manta™ and CNVnator™, respectively. The latter require 2 hours and 22 minutes longer cloud-based computation per sample than DRAGEN™. The recall (sensitivity) of DRAGEN™ was considerably superior for insertion SVs (average 27% with Manta™, 49% with DRAGEN™) and deletion CNVs (average 9% with CNVnator™, 88% with DRAGEN™, Table 9). Since the NIST reference sample contains only 33 CNVs, the latter values should not yet be regarded as general estimates of analytic performance. However, chromosomal microarray, the most widely used diagnostic test for CNVs only detected one deletion CNV in this sample (Chr 7:142,824,207-142,893,380del, 3% sensitivity), which was classified as benign. It should also be noted that the software used to calculate analytic performance for SV and CNV detection (Witty.Er), defines true positive matches more conservatively than in clinical diagnostic practice.

Automated Diagnosis of Genetic Diseases by Genome Sequencing.

Four further steps were needed for automated diagnosis of genetic diseases by WGS. Firstly, the patients' phenotypic features were automatically extracted from non-structured text fields in the electronic health record (EHR) using natural language processing (NLP, Clinithink™ Ltd.) through the date of enrollment for WGS. The analytic performance of NLP and detailed manual review were compared with EHRs of ten children who received WGS. NLP identified an average of 89.8 Human Phenotype Ontology™ (HPO™) features, including both exact matches and their hierarchical root terms (standard deviation (SD) 35.3, range 36-167; Table 10) per patient in ˜20 seconds. Compared with manual review, which took several hours per record, the precision (positive predictive value, PPV) of NLP was 0.80 (SD 0.15, range 0.57-0.97) and recall (sensitivity) was 0.90 (SD 0.14, range 0.50-0.98). The performance of NLP in extraction of clinical features from EHRs and reasons for identification of false positive clinical features have been previously described.

Secondly, for each patient, the extracted HPO terms observed in the patient at time of enrollment were compared with the known HPO™ terms for all 7,103 genetic diseases with known causative loci. Each genetic disease was assigned a likelihood of being the causative diagnosis based on the number of matching terms and their information content. Thirdly, the pathogenicity of each variant detected by WGS was calculated by database lookup, if previously described, and by prediction of variant consequence for the associated protein. Finally, a provisional genetic disease diagnosis was generated by rank ordering the integrated scores of phenotype similarity and diplotype pathogenicity. The provisional diagnosis contained none, one or a few genetic diseases. These four steps were integrated in three fully automated interpretation pipelines (InVitae MOON™, Fabric GEM™, and Illumina TruSight™ Software Suite, (TSS™)).

The diagnostic performance and reproducibility of this rWGS® system was compared, including the three interpretation pipelines, with blood samples from four affected children who had recently been diagnosed with a genetic disease by standard, clinical rWGS® and manual interpretation (Table 8, 11). The automated systems correctly diagnosed the four infants. The average rank of the correct diagnosis was 1, 2 and 1 for MOON™, GEM™ and TSS™, respectively, and the ranges were 1-1, 1-4, and 1-1, respectively (Table 12). The mean number of candidate diagnoses returned were 16.5, 8 and 3.5 for MOON™, GEM™ and TSS™, respectively, and time to execution 10.3, 41.5 and 224.3 minutes, respectively (Table 12). The TSS™ time included DRAGEN™ 3.7 processing time, whereas the others did not. The average time from blood sample to provisional diagnosis result was 13 hours 20.5 minutes, and fastest time was 13 hours 13 minutes (Table 8). In each case, MOON™ had the fastest computation time.

Development of an Information Resource for Genetic Diseases.

Manual interpretation is followed by writing a report of WGS results that includes information pertaining to the genetic diagnosis. This typically takes a genome analyst, genetic counselor, and laboratory director one or two hours. Automated interpretation tools do not yet provide written reports. To make automated WGS more generalizable, an information resource was developed to automatically provide such information to front-line physician teams (FIG. 9). First, the numerous, existing web-based information resources for genetic diseases were surveyed. Most were unstructured, incomplete, and not intended for use by front-line physicians. Datasets were obtained from Online Mendelian Inheritance in Man (OMIM™), Orphanet™, Genetics Home Reference (GHR™, now MedLinePlus™), DrugBank™ v5.0, the National Center for Advancing Translational Sciences resources (Inxight:Drugs™, Genetic and Rare Disease Information Center (GARD™), Medscape™, NORD's Rare Disease Database™, the National Center for BI resources (Gene™, ClinVar™, ClinicalTrials.gov™, GeneReviews™, and MedGen™), the Cochrane Database of Systematic Reviews™, and PubMed™.⁴⁶⁻⁵⁸ Transformation pipelines were built with the Konstanz Information Miner™ (KNIME) to match entries, normalize, and merge them.⁵⁹ Unifying gene definitions were from RefSeg™, and genetic disease definitions from mappings between OMIM™ and Orphanet™.^{46,47,60} OMIM™ identities were used except where there was only an Orphanet™ entry. Unifying HPO™ phenotypes were mapped to OMIM™, Orphanet™ and GARD™.^{46,47,61} A web resource, GTRx℠ (gtrx.rbsapp.net/) was developed to automatically display this information and link it to automated WGS results on a gene-by-gene basis (FIG. 9).

Development of an Electronic Acute Management Support System.

Clinical implementation of rWGS® has shown that rapid molecular diagnosis alone may be insufficient to improve outcomes in diseases with effective treatments that progress rapidly to severe morbidity or mortality if untreated. Front-line physicians are often unfamiliar with treatments for rare genetic diseases. Sub-specialist or multi-disciplinary consultation may materially delay treatment. Therefore, a virtual acute management guidance system for rare genetic diseases with effective treatments was developed, the Treatabolome™, that was integrated into the information resource described above (FIG. 9).

For common diseases, it would have been relatively straightforward to integrate DrugBank Plus™, Food and Drug Administration (FDA) indications, and additional resources such as InXight™ Drugs and ClinicalTrials.gov™. However, most drug treatments for rare childhood genetic diseases are prescribed off-label. Furthermore, specialized diets, dietary supplements, and surgeries, which are not subject to FDA review, are also critical components of treatment for rare childhood genetic diseases. Devices are another important class of intervention for children in ICUs. While devices are subject to FDA review, approvals are not tied to genetic disease diagnoses. Publicly available information resources were reviewed for rare childhood genetic disease interventions, including published clinical practice guidelines, OMIM™, Orphanet™, GHR™, GARD™, PubMed™, GeneReviews™, American College of Medical Genetics™ (ACMG™) Newborn Screening ACTion™ (ACT™) sheets, Acute Illness Materials™ developed by the New England Consortium of Metabolic Programs, and ActX™. A lack of broadly applicable instruments was discovered to measure rare genetic disease progression or outcomes, or orphan treatment effects, such as quality of life or real-world outcomes. Many genetic diseases lacked sufficient ground truth knowledge of variability in natural history if untreated, or relative effectiveness of standard of care treatments. Evidence of efficacy was generally short-term and from single-arm case reports or small case series. There was no consensus scheme for classification of the efficacy of treatments nor the quality of the evidence supporting efficacy. The best existing resource for treatment guidance for many different types of genetic diseases was GeneReviews™. However, it was unstructured and subject to many of these limitations. Content variability was compounded by review of each disease by a different set of experts. It did not review all childhood genetic diseases with effective treatments, and chapters were revised only every several years. It was necessary, therefore, to create a structured database of rare childhood genetic disease interventions that complied with the Findable, Accessible, Interoperable and Reusable (FAIR) guiding principles de novo.

In light of substantial shortcomings of normalized knowledge of genetic disease treatments, the narrowest scope for an electronic acute disease management support system was defined (FIG. 9). It was intended to guide initial, optimal treatment for critically ill children in ICUs at time of genetic disease diagnosis by rWGS®. It was limited to diseases with effective treatments and rapid progression in the absence of those treatments. It was designed for use by front-line intensivists, neonatologists and hospitalists during the time interval between return of rWGS® results and provision of authoritative subspecialist guidance or transfer to a tertiary or quaternary hospital. It was assumed that front-line physicians were unlikely to have treated a child with that disease in that setting before. It was also assumed that they would have limited genomic literacy, lack of familiarity with existing genetic disease information resources, and insufficient time to synthesize treatments by literature perusal. While limited in scope, interoperability with broader future use was sought.

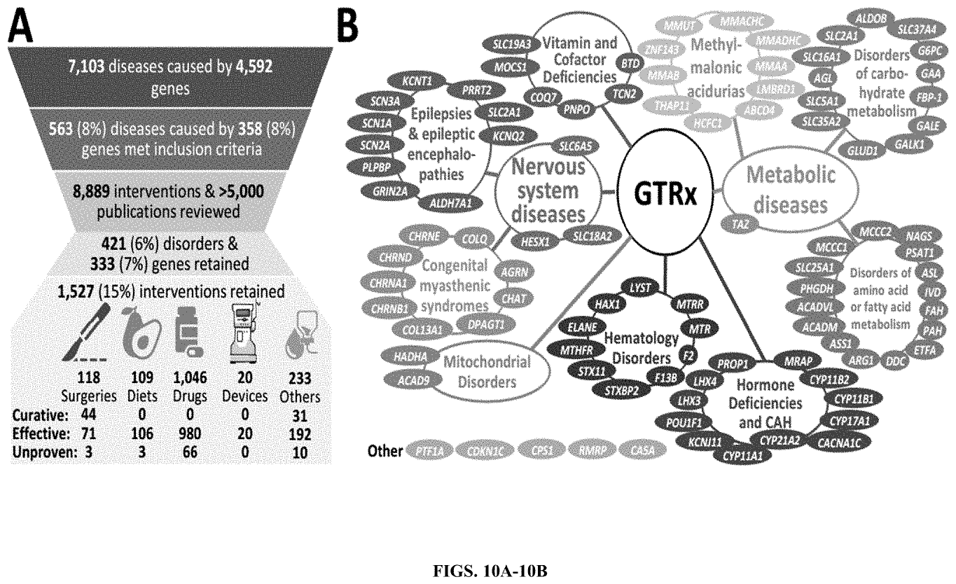

Second, 358 genes associated with 563 genetic diseases were identified, representing 8% of 7,103 single locus genetic diseases, that met the following criteria: acute, childhood presentations that were likely to lead to neonatal, pediatric or cardiovascular ICU admission; having somewhat effective treatments; high likelihood of rapid progression without treatment; and, diagnosable by rWGS® (FIGS. 9 and 10). They were identified by a survey of our clinical rWGS® experience in 3,500 cases, and from expanded newborn screening lists developed by several groups.

Third, the minimal data elements needed by front-line physicians upon receipt of an rWGS® result were determined. In the setting of a newly diagnosed genetic disease in a critically ill child, they needed to know the indicated interventions, optimal time to administration, efficacy, evidence for efficacy, contraindications, and natural history without treatment (Box 1). It was assumed that adequate resources existed to provide guidance about drug dosing, frequency, route of administration, drug-drug interactions or labelled contraindications.

Fourth, it was required that the virtual, acute disease management guidance system (GTRx^{sM}) was authoritative and consensus-driven. For each genetic disease, the full text of all MEDLINE/PubMed references that mentioned a drug, device, diet or surgery used to treat the disease using three artificial-intelligence based search engines (Mastermind™, Genomenon™; Rancho Biosciences™, Epam™ Systems, FIG. 9) were indexed. The resultant datasets were manually curated for relevance and specificity, and to extract the required data elements (data not shown). The manually curated datasets and links to the information resource were integrated into a custom Research Electronic Data Capture (REDCap™) survey for expert review (FIG. 9).⁷⁴ Each disease and intervention were reviewed by a panel of five highly experienced, pediatric biochemical geneticists to answer seven categorical questions (FIG. 9, Box 1). The first 15 genetic diseases and 200 associated interventions were independently reviewed by each expert. 52.8% of intervention reviews were concordant. Discordant responses were discussed virtually by the moderated panel (data not shown). After discussion, the panel agreed upon 189 (99%) of the first 190 (FIG. 9), and retained 84 interventions. There were three reasons for rejection of the remaining 106 nominated interventions: inadequate evidence for efficacy (25%, 27), incorrect treatment for that disorder (27%, 23), and insufficient specificity to warrant inclusion (19%, 20). Reviewers also examined the age category in which each intervention was suitable (neonate, infant, child), optimal time after diagnosis for initiation (hours, days/weeks, years), significant contraindications in subgroups of patients, efficacy of the intervention in that disease (curative, effective/ameliorative, still in trials/unproven), and level of published evidence for each intervention (authoritative clinical practice guideline, cohort study(ies), case report(s)). Consensus was reached for each question for each retained intervention. In addition, the experts identified appropriate consulting sub-specialists for each condition and emergency treatment notification flags, if any, that should accompany diagnostic reports.

Informed by experience with the first 15 disease genes, a total of 563 disorder-gene dyads underwent single primary, and secondary reviews by members of the same panel (FIG. 9). Primary reviews required 1-5 hours of effort by an expert medical geneticist, and secondary reviews required 1 hour of effort. Interventions lacking consensus were discussed by the five reviewers. Consensus was required for retention (data not shown). For disorders that reviewers or the moderator considered to require further input a final moderated review was performed by one or more pediatric subspecialists familiar with that disorder (FIG. 9). Examples of the latter included Timothy syndrome (cardiac electrophysiologist) and developmental epileptic encephalopathies (neonatal epileptologist). Review of 8,889 interventions and >5,000 publications by the expert panel led to retention of 421 (75%) disorders and 1,527 interventions (FIG. 10A), of which 118 (7.8%) were surgeries, 109 (7.2%) were diets or dietary supplements, 1,046 (68.8%) were medications, 20 (1.3%) were devices, and 233 (14.8%) were of other types (FIG. 10A). 75 (5.0%) retained interventions were considered curative, and 1,363 (90.6%) effective or ameliorative (FIG. 10A). Surgeries had the highest proportion of curative interventions (37.6%). The disease genes mapped to many organ systems and pathologic mechanisms (FIG. 10B).

The retained interventions and qualifying statements were incorporated into the GTRx℠ information resource as a prototypic acute management guidance system for genetic diseases that meets FAIR principles (FIG. 9,10, gtrx.radygenomiclab.com).

Physician Perception of the Utility of GTRx℠.

The clinical utility, ease of use and ease of comprehension of the GTRx℠ information resource and management guidance was evaluated by nine senior neonatologists and pediatric intensivists who were not involved in its design or development. On a 10-point Likert scale, their median perception as to whether they would use GTRx℠ was 9, ease of use was 9, and the utility of the information was 6 (data not shown). GTRx℠ was perceived to meet clinical needs somewhat well. In response to specific feedback, the GTRx℠ website was modified to increase ease of use, clarity, and to elicit ongoing feedback.

Performance of the System for Automated Provisional Diagnosis and Electronic Acute Management Support.

In four retrospective cases, the automated pipeline and electronic acute management support system identified the correct diagnosis in 13:13-13:27 hours (Table 8). An independent physician evaluated the accuracy of the treatment guidance from the virtual acute management support system. In each case, the interventions were assessed to be correct and complete (Table 8, Table 10).

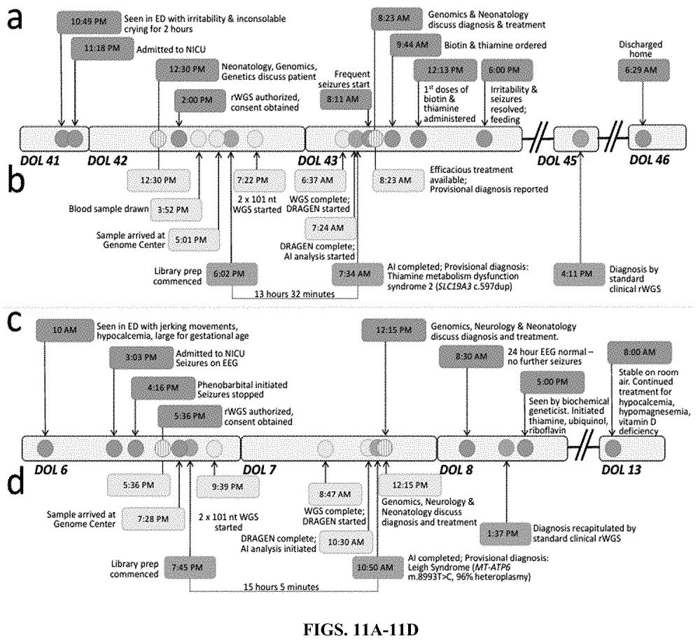

The performance of the 13.5-hour system for automated provisional diagnosis and the GTRx℠ electronic acute management support system were prospectively compared with the fastest standard clinical methods in three infants (Table 8, FIG. 11). The first prospective case, AH638, was a 6-week-old male admitted to the neonatal ICU with extreme irritability and inconsolable crying. Brain magnetic resonance imaging revealed widespread, symmetric hypodense lesions. Electroencephalography (EEG) revealed frequent seizures. The proband's elder sister died nine years earlier, at 11 months of age, after presenting at the same age with the same symptoms and findings. WGS was not available at that time, and she died of progressive developmental epileptic encephalopathy without an etiologic diagnosis. His parents were first cousins. The prototypic methods provided a provisional diagnosis in 13 hours and 32 minutes. The diagnosis was autosomal recessive thiamine metabolism dysfunction syndrome 2, biotin- or thiamine-responsive type (Online Mendelian Inheritance in Man™ (MIM™) #607483, omim.org/entry/607483) associated with a pathogenic, homozygous, frameshift variant in the thiamine transporter 2 gene (SLC19A3 c.597dup, p.His200fs, ncbi.nlm.nih.gov/clinvar/variation/533549/?oq=SLC19A3[gene]+AND+c.597dupT[varname]+& m=NM 025243.4(SLC19A3):c.597dup %20(p.His200fs)). The provisional diagnosis was immediately communicated to the neonatologist of record. Effective treatments (biotin and thiamine supplements) were initiated within 3 hours of diagnosis. He responded to treatment and was alert, tranquil, and bottle feeding within six hours of treatment. Standard clinical rWGS® methods recapitulated the diagnosis in 42 hours and 39 minutes. He had no further seizures and was discharged home after 3 days. At fifteen months of age, he has had no further seizures. He is making developmental progress but has delayed motor and language development.

The second patient, CSD59F, a male, was admitted to the neonatal ICU on day of life 6 after his mother noticed abnormal, jerking movements (Table 8, FIG. 11). EEG disclosed frequent seizures. He had hypocalcemia (6.1 mg/dL, reference range 7.6-10.4 mg/dL) and hyperphosphatemia (11.2 mg/dL, reference range 4.3-9.3 mg/dL). The prototypic methods yielded a provisional diagnosis of Leigh syndrome (MIM #256000, omim.org/entry/256000) in 15 hours and 5 minutes. Peripheral blood DNA had de novo 96% heteroplasmy (1351/1402 reads) for a well-established, pathogenic variant in the mitochondrial ATP synthase subunit 6 gene (MT-ATP6 m.8993T>C, p.Leu156Pro, ncbi.nlm.nih.gov/clinvar/variation/9642/?oq=MT-ATP6[gene]+AND+m.8993T %3EC[varname]+&m=NC_012920.1:m.8993T %3EC). Leigh syndrome is associated with infantile seizures. The provisional diagnosis of Leigh syndrome was immediately communicated to the neonatologist of record. A heterozygous variant of uncertain significance was also identified in the SET domain-containing protein 1A gene (SETD1A c.4105G>A, p.Gly1369Arg, ncbi.nlm.nih.gov/clinvar/variation/834092/?oq=SETD1A[gene]+AND+c.4105G %3EA[varname]+&m=NM_014712.3(SETD1A):c.4105G %3EA %20(p.Gly1369Arg)). Pathogenic variation in SETD1A is associated with autosomal dominant, Early-Onset Epilepsy with or without developmental delay (MIM #618832, omim.org/entry/618832). This finding was not reported provisionally. Standard clinical rWGS® methods recapitulated these findings in 42 hours and 5 minutes, and a final report was issued of both findings. Seizures remitted with phenobarbital. He was seen by a subspecialist in mitochondrial diseases within 48 hours of admission, and initiated on thiamine, ubiquinol and riboflavin supplementation. He was discharged in stable condition with no further seizures on day of life 23.

The third patient, CSD709, a male, was admitted to the neonatal ICU on the first day of life with respiratory failure, lactic acidosis, encephalopathy, hypotonia, multiple congenital anomalies (short long bones in the upper and lower limbs, posteriorly rotated ears, dysmorphic knees, and congenital heart disease (pulmonary artery stenosis, pulmonary arterial hypertension, aortic valve stenosis, and right ventricular hypertrophy))(Table 8). rWGS® was completed in 14 hours and 14 minutes by the prototypic methods but did not yield a provisional diagnosis. Standard clinical rWGS® methods completed in 27 hours and 46 minutes. Both disclosed a heterozygous, likely pathogenic, SNV in a disintegrin and metalloproteinase with thrombospondin motifs-like protein 2 (ADAMTSL2 c.338G>T, p.Arg113Leu, ncbi.nlm.nih.gov/clinvar/variation/1326072/?oq=ADAMTSL2[gene]+AND+c.338G %3ET[varna me]+&m=NM_014694.4(ADAMTSL2):c.338G %3ET %20(p.Arg113Leu)) that had previously been reported in patients with geleophysic dysplasia (MIM #231050, omim.org/entry/231050?search=231050&highlight=231050) as a compound heterozygous or homozygous change. The variant call file (vcf) did not contain a second variant in ADAMTSL2. However, ADAMTSL2 is located in a region that is affected by segmental duplication. Manual inspection of aligned ADAMTSL2 reads revealed a second heterozygous, likely pathogenic variant (c.1851C>A, p.Cys617Ter, ncbi.nlm.nih.gov/clinvar/variation/1326007/?oq=ADAMTSL2 [gene]+AND+c.1851C %3EA[var name]+&m=NM_014694.4(ADAMTSL2):c.1851C %3EA %20(p.Cys617Ter)). Both variants were confirmed to be in trans by orthogonal methods and a diagnosis of geleophysic dysplasia was reported after 14 days.

Discussion

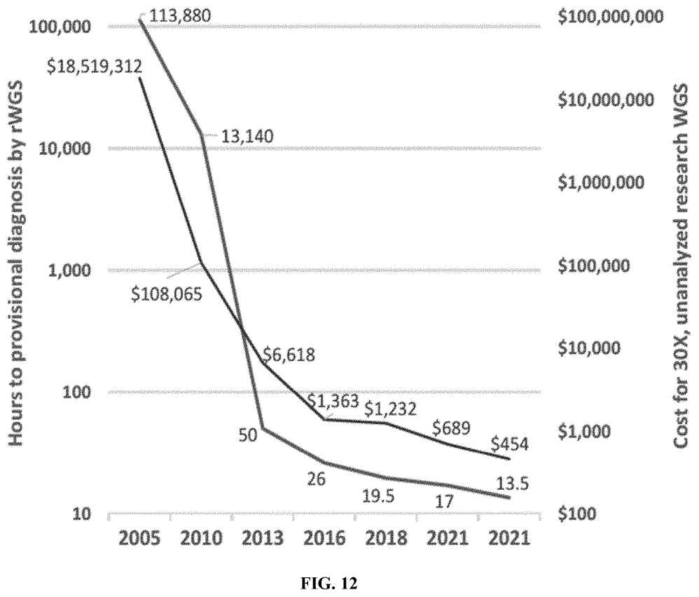

The cost and turnaround time of WGS have decreased dramatically since its advent 15 years ago (FIG. 12). The first human genome took 13 years to complete. Described herein is the performance of a 13.5-hour, autonomous system for genetic disease diagnosis by rapid WGS and virtual, specific management guidance. This is the fifth reduction in the minimal time to diagnosis by WGS since 2012. While this manuscript was under review, a 7-hour, method for genetic disease diagnosis by long-read WGS was published. The rationale for continuing to pursue faster diagnosis was strikingly exemplified in the first infant to receive 13.5-hour WGS. He was diagnosed in 13 hours and 32 minutes with a disorder that is both treatable and extremely rapidly progressive. Had his diagnosis been delayed until the standard rWGS® result (42.5 hours) he would likely have had significant, permanent neurologic damage. In contrast, his sister died without an etiologic diagnosis, and thus, without effective treatment. The experience in this family was not unique. Since it is not possible to determine a priori which cases require such rapidity, the general practice has been to provide the fastest turnaround possible for all critically ill infants and children or those with rapid clinical progression in ICUs and who have diseases of unknown etiology. At current volume of ˜100 cases per month, our median turnaround time for critical cases is 30-36 hours. In clinical production in three cases, it was found that these methods have reduced this by a factor of two.

There is now strong evidence that diagnosis of genetic diseases by rWGS® improves outcomes of infants and children in regional ICUs, irrespective of presentation or health system. As a result, diagnostic rWGS® is being implemented for such children in England, Wales, and Germany, by Anthem/BlueCross/BlueShield in the USA, and by Medicaid in California and Michigan. Scalability of rWGS® in routine practice is, therefore, as important as turnaround time. The 13.5-hour system for genetic disease diagnosis incorporated several innovations that enhance scalability and reproducibility. These included automated interpretation, which is extremely important since there are insufficient molecular pathologists, molecular laboratory directors, genetic counselors and clinical genome analysts for manual interpretation of results from all of the children for whom rWGS® is being implemented. As sequencing costs decrease (FIG. 12), manual interpretation and reporting are becoming the largest component of the expense of diagnostic rWGS®. Herein, three, cloud-based methods for autonomous genetic disease diagnosis were compared, providing the opportunity for cross checking of results. The only requirements for implementation of this system are an EHR, internet access, and a regional diagnostic lab with a suitable sequencer. A cloud-based, automated interpretation that is supervised by a laboratory director and supplemented with centralized, manual interpretation for edge cases is envisaged. The diagnostic performance of the automated interpretation system GEM™ was recently examined in 193 children with suspected genetic diseases. In 92% of cases, GEM™ ranked the correct gene and variant in the top two calls, including structural variant diagnoses. However, to date the full 13.5-hour system has been evaluated only in four retrospective and six prospective cases. Further studies are needed for clinical validation, such as reproducibility, performance with all patterns of inheritance, examination of the relative diagnostic performance of automated methods compared with traditional manual interpretation, and to understand the proportion of edge cases.

Another innovation of the system described herein was ability to diagnose genetic diseases associated with most major classes of genomic variants. Hitherto, diagnostic speed was achieved at the expense of limitation to small (nucleotide) variants, which represent 75-80% of genetic disease diagnoses. Here, methods for library preparation, variant calling, and automated interpretation were used that enabled structural and copy number variant (SV, CNV) diagnoses with improved performance. It should be noted, however, that recall (sensitivity) for SVs and CNVs remain a weakness of short read sequencing (range 49%-88%). The consequences of this for genetic disease diagnosis is not yet known. Further studies are needed to compare the diagnostic performance of these methods versus hybrid methods with short read sequencing and complementary technologies, such as long-read sequencing and optical mapping.

Finally, the 13.5-hour system featured a virtual clinical decision support system, GTRx℠ to decrease variability or delayed implementation of specific treatment following diagnosis of rare genetic conditions. Hitherto, use of rWGS® has been almost entirely in ICUs in regional, academic, tertiary, or quaternary centers with specialist neonatologists and access to a full range of subspecialist consultants. Lack of familiarity with management of specific, rare genetic diseases leads to delays in consultation and missed opportunities for treatment that defeat the goal of rapid diagnosis. GTRx℠ was developed both to increase the proportion of children who receive optimal, immediate treatment and to facilitate broader use of rWGS®, such as in local birthing hospitals staffed by front-line neonatologists. In California, for example, while 18% of newborns are admitted to level II and III NICUs in community birthing hospitals, only 2% of newborns are transferred to regional, level IV neonatal intensive care units. Transfers are often delayed since there is a strong desire to provide care for the newborn at the same location as his or her mother, and it is often not readily apparent that subspecialist care is required. In many regions of the US, geographic isolation limits transfer. GTRx℠ adheres to the technical standards developed by the ACMG for diagnostic genomic sequencing. The most recent guidelines suggest the addition of references to treatments in reports of genes associated with a treatable genetic disorder.

The extent to which rare genetic diseases did not have organized management guidance was surprising. For many, the mechanism of disease remained unclear, and the treatment literature comprised only case reports or small case series. Most interventions were off label. Furthermore, no general schema existed whereby to classify the relative efficacy of interventions for specific genetic disorders nor the quality of the evidence for efficacy. Methods to extract and transform treatment data from the literature were developed. A categorical framework for nomenclature, efficacy, evidence, indicated population, immediacy of initiation of treatment and warnings were developed. Tiered reviews were used, facilitated by artificial intelligence and REDCap™, and expert consensus to retain efficacious interventions. The resultant prototypic acute management guidance tool and information resource, GTRx℠, was intended for use by front-line neonatologists and intensivists upon receipt of results of rWGS® for children under their care in ICUs. It did not require genomic or genetic literacy. Version 1 of GTRx℠ covers 457 genetic disorders that cause infant or early childhood ICU admission and that have somewhat effective, time-delimited treatments. GTRx℠ is publicly available for research use at present.

Version 1 of GTRx℠ does not cover all genetic diseases of known molecular cause, that can be diagnosed by rWGS®, can lead to ICU admission in infancy, and have effective treatments. In addition, the literature related to disease treatments is continually being augmented. While pediatric geneticists were optimal subspecialists for initial review of disorders and interventions, many would benefit from additional sub- and super-specialist review. In addition, recent evidence supports the use of rWGS® for genetic disease diagnosis and management guidance in older children in pediatric ICUs. There are several, additional, complementary information resources that would enrich GTRx℠, such as ClinGen™, the Genetic Test Registry™, and Rx-Genes™. Finally, there are many clinical trials of new interventions for infant-onset, severe genetic disorders, particularly genetic therapies. For disorders without a current effective treatment, it is desirable to include links to enrollment contacts for those clinical trials.

Currently, pathogenicity guidelines help molecular laboratory directors standardize how many and which genome findings to report. GTRx℠ will help standardize the reporting of variants of uncertain significance (VUS), which, at present, is predicated on the goodness of fit of the patient's presentation and the phenotype associated with the variant containing gene. In the setting of GTRx℠, VUS reporting will be further prioritized by the availability of an effective treatment for the associated disease, akin to variant tiering in oncology⁹³. The GTRx℠ information resource will simplify the writing of rWGS® reports, extending the ability to automate diagnosis. Thus, for each automated WGS result, GTRx℠ provides access to information about each genetic disease, including inheritance, incidence, symptoms and signs, progression, complications and outcomes, and the causal gene, including function, and mechanism of disease.

As genomic literacy and experience evolves, physicians increasingly wish to reinterpret findings themselves, dynamically adjusting the scope of review on a case-by-case basis. In the longer term, automated genome interpretation and virtual management guidance have the potential to empower dynamic physician re-analysis. It is envisaged GTRx℠ will evolve into a virtual physician assistant, equipping physicians to dynamically explore the goodness of fit of observed and various candidate disease phenotype sets. Where associated diplotypes are incomplete or include variants of uncertain significance, GTRx℠ will allow ordering of confirmatory tests. GTRx℠ will also assist physicians in decision making with regard to a possible trial of treatment for a potential diagnosis, guided by the risk: benefit ratio. This is particularly important for critically ill patients where a genetic etiology is strongly suspected but genome findings are insufficient for strict molecular diagnosis. GTRx℠ will also assist front-line physicians to communicate with families about the ramifications of rare genetic disease diagnoses. GTRx℠ is part of a major trend in medicine—adding artificial intelligence to physician competency to deliver “high-performance medicine”.

In summary, described herein is a 13.5-hour prototypic system for automated genetic disease diagnosis and acute management guidance. The system was designed to expand the use of rWGS® by front-line physicians caring for critically ill infants and children in ICUs. At present, the system is prototypic and encompasses only ˜500 genetic diseases that progress rapidly, and for which effective treatments are available. Upon validation of clinical utility, expansion of the system to all genetic diseases and to dynamic filtering is envisaged, enabling front-line physicians to play a much more active role in evaluating potential genetic etiologies and their consequent therapies in their patients.

### Figure Legends

FIG. 8. Flow diagrams of the technological components of a 13.5-hour system for automated diagnosis and virtual acute management guidance of genetic diseases by rWGS®. Innovations described herein are indicated by orange boxes A. The order and duration of laboratory steps and technologies. EHR: Electronic Health Record, EDTA: EthyleneDiamineTetraAcetic acid, gDNA: genomic DeoxyriboNucleic Acid; PCR: Polymerase Chain Reaction, QA: Quality Assurance, nt: Nucleotide, SNV: Single Nucleotide Variant, indel: insertion-deletion nucleotide variant, SV: Structural Variant, CNV: Copy Number Variant, GTRx℠: Genome-to-Treatment. B. Diagram of the information flow from order placement in the EHR to return of diagnostic results together with specific management guidance for that genetic disease. rWGS® Portal: Custom software system for rWGS® ordering, accessioning, chain-of-custody, and return of results (v.3.2). LIMS: Custom laboratory information management system for rWGS®, short tandem repeat profiling, confirmatory testing (Sanger sequencing and Multiplex Ligation-dependent Probe Amplification), and inventory management (L7 informatics). IR: Information resource, *: HL7/FHIR or Continuity of Care Documents, †: JSON. ‡: bcl, □: vcf.

FIG. 9. Flowchart of the development of GTRx℠, a virtual system for acute management guidance for rare genetic diseases. Phase 1—Compilation of a comprehensive gene-genetic disease list for severe, childhood-onset conditions in which an established treatment was available. Phase 2, integration of 13 information resources pertaining to rare genetic diseases. Phase 3, development of the GTRx℠ web resource containing the integrated information resources. Phase 4, automated, artificial intelligence (AI)-based searching and manual curation of published evidence of treatments for each condition by three companies. Phase 5, development of a custom REDCap™ system for structured assessment of genes, disorders, and therapeutic interventions. Phase 6a, independent manual review of curated interventions and assertions for the first 15 pilot gene-disease pairs by five experts. Phase 6b, primary and secondary reviews of the remaining gene-disease pairs. Phase 7, round-table discussion of records lacking consensus. Phase 8, upload of retained consensus records to the GTRx℠ web resource.

FIG. 10. GTRx℠ disease, gene, and literature filtering, and final content. A. A modified PRISMA flowchart showing filtering steps and summarizing results of review of 563 unique disease-gene dyads herein⁸⁴. B. Genetic disease types and disease genes featured in the first 100 GTRx℠ genes reviewed herein.

FIG. 11. Clinical (a and c, dark blue circles) and diagnostic timelines (b and d, light blue circles) of infants AH638 (a and b) and CSD59F (c and d), who received both standard, clinical rWGS® and the 13.5-hour methods. ED: Emergency Department. EEG: Electroencephalogram. AI: Artificial intelligence. DOL: Day of life. Circles with vertical lines indicate interactions between neonatology, genomics, and biochemical genetics.

FIG. 12. Decreasing cost of research WGS (red line) and time to provisional diagnosis of rapid, clinical WGS (blue line) of WGS, 2005-2021. Source data are provided as a Source Data file.

### Supplementary Materials (Example 2)

Tables

TABLE 8
Analytic performance, reproducibility, and duration of the major steps in automated diagnosis of genetic diseases
by accelerated rWGS ®. Analytic and diagnostic reproducibility were examined for sample 362 from 19.5-hour rWGS ® (16),
reference samples NA12878 and NA24385, four retrospective samples/diagnoses (AG928/Hereditary fructose intolerance (compound
heterozygous, pathogenic (P) SNVs in aldolase B [ALDOB c.448G > C, c.524C > A]); AG366/Ornithine transcarbamylase deficiency
(hemizygous, de novo, P, SNV in ornithine transcarbamylase [OTC c.275G>A]); AF414/Propionic acidemia (homozygous, likely
pathogenic (LP) indel in a-subunit of propionyl-CoA carboxylase [PCCA c. 1899+4 1899+7del]); A1003/Developmental and epileptic
encephalopathy 11 (heterozygous, de novo, LP SNV in the a2-subunit of the voltage-gated sodium channel [SCN2A c.4437G > C]), and
three prospective samples (AH638/Thiamine metabolism dysfunction syndrome 2 (homozygous, P, frame-shift variant in solute
carrier 19, member 3 [SLC19A3 c.597dup]), CSD59F (heteroplasmic, P, SNV in the mitochondrial ATP synthase 6 gene [MT-ATP6
m.8993T>C]), and CSD709/ Geleophysic dysplasia (compound heterozygous SNVs in ADAMTS-like 2 [ADAMTSL2 c.338G>T and
c.1851C>A]), which received rWGS® both with the 13.5-hour method (Herein) and standard, singleton or trio, clinical rWGS ®
(Std)(Table 11). Ref.16: Reference 16. Sample 12878: Sample NA12878. ID: Identification. Here: Herein. 1°/2° analysis time:
Conversion of raw data from base call to FASTQ format, read alignment to the reference genomes and variant calling. Tertiary
analysis: Time of automated interpretation to provisional diagnosis (most rapid of three systems run in parallel (MOON ™, Illumina
TruSight ™ Software Suite and GEM). SV and CNV detection methods: MC: Manta and CNVnator. [symbol] : DRAGEN™ version 3.7.
D3.5: DRAGEN ™ version 3.5.3. MIM ™: Mendelian inheritance in man. Nt: Nucleotide. Gene symbols are shown in italics. Variant
section headers are shown in bold.

| Sample | 362 | 12878 | NA24385 | | | AG928 | AG366 | AF414 | AI003 |
|---|---|---|---|---|---|---|---|---|---|
| Run | Ref. 16 | 927 | 929 | 930 | 1018 | 1020 | 1204 | 1208 | 1218 |
| Sample & Run | DNA / Analytic Performance | | | | | Blood/Retrospective | | | |
| Type | | | | | | | | | |
| Diagnosis (Gene) | None | | | | | | | | ALDOB |
| rWGS Methods | Ref. 16 | Herein | | | | | | | Herein |
| SV & CNV ID | None | [symbol] | MC | [symbol] | MC | [symbol] | MC | [symbol] | [symbol] |
| Method | | | | | | | | | |

Length of steps (min)

| Sample Prep. | 151 | 50 | 45 | | 41 | | 50 | | 74 | 71 | 69 | 67 |
|---|---|---|---|---|---|---|---|---|---|---|---|---|
| Time | | | | | | | | | | | | |
| Sequencing Time | 932 | 667 | 667 | | 666 | | 673 | | 674 | 667 | 683 | 675 |
| 10/20 Analysis | 62 | 48 | 191 | 45 | 181 | 46 | 194 | 48 | 42 | 55 | 37 | 38 |
| Time | | | | | | | | | | | | |
| Tertiary Analysis | n.a. | n.a. | n.a. | n.a. | n.a. | n.a. | n.a. | n.a. | 10 | 14 | 13 | 13 |
| Total Time to | 1,145 | 765 | 903 | 757 | 888 | 753 | 917 | 761 | 800 | 807 | 802 | 793 |
| Result | | | | | | | | | | | | |

Sequence Metrics

| | | | | | | | | | |
|---|---|---|---|---|---|---|---|---|---|
| Trimmed Yield | 149 | 192 | 178 | 186 | 189 | 165 | 176 | 80 | 135 |
| (Gigabases) | | | | | | | | | |
| Reads with | 90.7% | 90.5% | 88.7% | 90.8% | 91.3% | 89.2% | 91.2% | 92.5% | 87.3% |
| Quality Score >30 | | | | | | | | | |
| Error rate | n.a. | 0.17% | 0.21% | 0.17% | 0.14% | 0.19% | 0.16% | 0.14% | 0.29% |
| Reads Mapped | 98.9% | 96.7% | 96.8% | 96.8% | 97.2% | 96.0% | 96.9% | 89.0% | 94.8% |
| Duplicate Reads | 8.5% | 11.6% | 10.8% | 12.9% | 13.9% | 15.2% | 15.5% | 23.2% | 14.5% |
| Mean Insert Size | 345 | 395 | 438 | 449 | 445 | 440 | 426 | 496 | 468 |
| (Nt) | | | | | | | | | |
| Average Genome | 47.5 | 52.3 | 49.1 | 49.9 | 50.5 | 44.5 | 47.44 | 19.46 | 36.7 |
| Coverage | | | | | | | | | |
| MIM genes w. | 95.8% | 97.6% | 96.4% | 96.7% | 97.1% | 94.9% | 95.8% | 4.2% | 92.2% |
| >10X coverage of | | | | | | | | | |
| all coding domain | | | | | | | | | |
| Nt. | | | | | | | | | |

Variant Metrics

| Nt Variants | 4,733 | 4,834 | 4,838 | 4,838 | 4,837 | 4,857 | 3,789 | 3,904 | 4,851 |
|---|---|---|---|---|---|---|---|---|---|
| (1,000s) | | | | | | | | | |
| Variants passing | 96.8% | 98.9% | 99.1% | 99.1% | 99.0% | 99.0% | 99.0% | 98.4% | 98.6% |
| Quality Metrics | | | | | | | | | |
| Coding Domain | 0.58% | 0.51% | 0.52% | 0.52% | 0.52% | 0.53% | 0.52% | 0.52% | 0.51% |
| Variants | | | | | | | | | |
| Nt insertions & | 17.5% | 19.7% | 19.7% | 19.7% | 19.6% | 19.5% | 19.6% | 18.9% | 19.4% |
| deletions | | | | | | | | | |
| Transition/ | 2.02 | 2.03 | 2.02 | 2.02 | 2.02 | 2.03 | 2.03 | 2.03 | 2.03 |
| Transversion | | | | | | | | | |
| Ratio | | | | | | | | | |
| Sample | AH638 | | CSD59F | | CSD709 | | | | |
| Run | 1026 | 1027 | 477 | 480 | 478 | 479 | | | |
| Sample & Run | Blood/Prospective | | | | | | | | |
| Type | | | | | | | | | |
| | | | | | | MT-ATP6, | | | |
| Diagnosis (Gene) | OTC | PCCA | SCN2A | SLC19A3 | | SETD1A | | AdamtsL2 | |
| rWGS Methods | Herein | | | Here | Std | Here | Std | Here | Std |
| SV & CNV ID | [symbol] | [symbol] | [symbol] | [symbol] | D3.5 | [symbol] | D3.5 | [symbol] | D3.5 |
| Method | | | | | | | | | |

Length of steps (min)

| | | | | | | |
|---|---|---|---|---|---|---|
| Sample Prep. | 80 | 1,233 | 90 | 265 | 90 | 265 |
| Time | | | | | | |
| Sequencing Time | 676 | 1,067 | 687 | 1,050 | 687 | 1,050 |
| 10/20 Analysis | 47 | 173 | 44 | 185 | 56 | 220 |
| Time | | | | | | |
| Tertiary Analysis | 10 | 87 | 12 | 126 | 21 | 131 |
| Total Time to | 812 | 2,560 | 833 | 1,626 | 854 | 1,666 |
| Result | | | | | | |

Sequence Metrics

| | | | | | | |
|---|---|---|---|---|---|---|
| Trimmed Yield | 187 | 162 | 182 | 144 | 174 | 153 |
| (Gigabases) | | | | | | |
| Reads with | 90.5% | 92.6% | 90.9% | 89.8% | 90.1% | 89.3% |
| Quality Score >30 | | | | | | |
| Error rate | 0.17% | 0.15% | 0.14% | 0.14% | 0.17% | 0.16% |
| Reads Mapped | 96.2% | 99.1% | 96.1% | 99.1% | 95.5% | 98.6% |
| Duplicate Reads | 13.7% | 11.4% | 15.8% | 10.4% | 14.9% | 13.6% |
| Mean Insert Size | 465 | 423 | 491 | 467 | 502 | 460.5 |
| (Nt) | | | | | | |
| Average Genome | 49.1 | 45.7 | 46.9 | 40.7 | 45.1 | 41.5 |
| Coverage | | | | | | |
| MIM genes w. | 90.1% | 95.5% | 94.6% | 94.5% | 94.7% | 94.6% |
| >10X coverage of | | | | | | |
| all coding domain | | | | | | |
| Nt. | | | | | | |

Variant Metrics

| | | | | | | |
|---|---|---|---|---|---|---|
| Nt Variants | 4,691 | 4,690 | 4,852 | 4,852 | 4,916 | 4,910 |
| (1,000s) | | | | | | |
| Variants passing | 98.9% | 98.9% | 99.0% | 98.9% | 98.9% | 98.9% |
| Quality Metrics | | | | | | |
| Coding Domain | 0.52% | 0.52% | 0.52% | 0.52% | 0.53% | 0.53% |
| Variants | | | | | | |
| Nt insertions & | 19.6% | 19.6% | 19.7% | 19.7% | 19.7% | 19.7% |
| deletions | | | | | | |
| Transition/ | 2.03 | 2.03 | 2.03 | 2.03 | 2.03 | 2.03 |
| Transversion | | | | | | |
| Ratio | | | | | | |

TABLE 9
Comparison of the analytic performance of standard, clinical rWGS ® and the 13.5-hour method. The analytic
performance of DRAGENTM v.3.7 for SNVs and indels was compared with DRAGENTM v2.5, the prior method (16), in reference
samples NA12878 and NA24385, using NIST benchmark genotypes. The analytic performance of DRAGEN ™ v.3.7 for SVs and
CNVs was compared with Manta and CNVnator ™ (MC) in triplicate libraries in reference sample NA24385, using NIST benchmark
genotypes. SV and CNV evaluations used Witty.Er (What is true, thank you, earnestly) [75], with default settings except event
reporting [—em cts]). SVs were of size > 50 nt and CNVs >10 kb.

| | | Variant | NA12878 | | Variant | NA24385 | |
|---|---|---|---|---|---|---|---|
| Variant Type | Performance Metric | Number | v.2.5 | [symbol] | Number | MC | [symbol] |
| SNV | Precision | 3,258,654 | 99.8% | 99.9% | 3,440,606 | n.a. | 99.7% |
| | Recall | | 99.7% | 99.9% | | n.a. | 99.3% |
| indel | Precision | 490,488 | 99.0% | 99.6% | 553,766 | n.a. | 99.4% |
| | Recall | | 95.5% | 99.4% | | n.a. | 98.6% |
| SV deletion | Precision | n.a. | n.a. | n.a. | 4,203 | 91.7% | 97.1% |
| | Recall | | n.a. | n.a. | | 57.3% | 61.7% |
| SV insertion | Precision | n.a. | n.a. | n.a. | 5,444 | 99.0% | 98.4% |
| | Recall | | n.a. | n.a. | | 27.4% | 49.3% |
| CNV deletion | Precision | n.a. | n.a. | n.a. | | 83.3% | 100.0% |
| | Recall | | n.a. | n.a. | 5 | 9.1% | 87.9% |

TABLE 10
Precision and recall of phenotypic features extracted by clinical natural language processing (CNLP) from EHRs
in 10 children with genetic diseases. Precision = tp/tp + fp. Recall=tp/tp + fn. Abbreviations: AD: Autosomal Dominant; AR: Autosomal
Recessive; DN: de novo', P: Pathogenic; LP: Likely Pathogenic; S: Singleton; T: Trio; I: Inherited; U: undetermined; OMIM ™: Online
Mendelian Inheritance in Man; Inh: Inheritance.

| | S | WES | | | | | | | |
|---|---|---|---|---|---|---|---|---|---|
| | or | or | | Affected | OMIM | | DN | | |
| Family | T | WGS | Disease | Gene | ID | Inh | or I | Variant 1 (V1) | Variant 2 (V2) |
| 201 | T | WES | Prader Willi | 15q11- | 176270 | AD | DN | Chr15:23684685- | |
| | | | Syndrome | q13 del | | | | 26108259del | |
| 205 | T | WGS | Dursun | G6CP3 | 612541 | AR | I | c.207dupC, | c.199)_218 + 1delCTCAACCTC |
| | | | Syndrome | | | | | p.IIe70HisfsTer17 | ATCTTCAAGTGG |
| 213 | S | WGS | Visceral | NODAL | 270100 | AD | I | c.778G > A, p.Gly260Arg | |
| | | | Heterotaxy 5 | | | | | | |
| 233 | T | WGS | Tuberous | TSC1 | 191100 | AD | DN | c.1498C > T, | |
| | | | Sclerosis 1 | | | | | p.Arg500Ter | |
| 243 | T | WGS | Pyridoxine | ALDH7A1 | 266100 | AR | I | c.328C > T, | c.1279G > C, p.Glu427Gln |
| | | | dependent | | | | | p.Arg110Ter | |
| | | | seizures | | | | | | |
| 6094 | T | WGS | Argininosuccinic | ASL | 207900 | AR | I | c.706C > T, | c.706C > T, p.Arg236Trp |
| | | | inic aciduria | | | | | p.Arg236Trp | |
| 6098 | T | WGS | Gaucher | GBA | 230800 | AR | I | c.1503C > G, | c.1448T > C, p.Leu483Pro |
| | | | disease | | | | | p.Asn501Lys | |
| 6108 | T | WGS | Tuberous | TSC2 | 613254 | | DN | c.935_936delTC, | |
| | | | Sclerosis 2 | | | | | p.Leu312GlnfsTer25 | |
| 7003 | T | WGS | Epileptic | SCN1A | 607208 | | DN | c.5555T > C, | |
| | | | Encephalopathy 6 | | | | | p.Met1852Thr | |
| 7004 | T | WGS | Hypertrophic | MYH7 | 192600 | | I | c.746G > A, | |
| | | | cardiomyopathy 1 | | | | | p.Arg249Gln | |
| Average | | | | | | | | | |
| Standard Deviation | | | | | | | | | |
| | | V1 | V2 | | | | | | |
| | | P/ | P/ | DOL data | | Consan- | CNLP | CNLP | CNLP |
| | Family | LP | LP | extract | Sex | guinity | Features | Precision | Recall |
| | 201 | | | 4 | [symbol] | U | 89 | 0.53 | 0.95 |
| | 205 | P | P | 2 | [symbol] | No | 94 | 0.93 | 0.95 |
| | 213 | | | 3 | [symbol] | U | 89 | 0.90 | 0.98 |
| | 233 | | | 5 | [symbol] | No | 167 | 0.90 | 0.98 |
| | 243 | | | 6 | [symbol] | No | 36 | 0.97 | 0.50 |
| | 6094 | P | P | 7 | [symbol] | Yes | 55 | 0.85 | 0.87 |
| | 6098 | | | 215 | [symbol] | No | 112 | 0.92 | 0.94 |
| | 6108 | | | 4 | [symbol] | No | 86 | 0.76 | 0.94 |
| | 7003 | | | 424 | [symbol] | U | 67 | 0.81 | 0.93 |
| | 7004 | | | 5171 | [symbol] | U | 99 | 0.68 | 0.96 |
| | Average | | | | | | 89.4 | 0.79 | 0.90 |

| | | | |
|---|---|---|---|
| Standard Deviation | 35.3 | 0.15 | 0.14 |

TABLE 11
Characteristics of four retrospective cases used to test performance of the 13.5 hour automated sequencing and
interpretation pipeline. Abbreviations: AD: Autosomal Dominant; DN: de novo', P: Pathogenic; LP: Likely Pathogenic; M: Male; F:
Female; S: Singleton; T: Trio; I: Inherited; XL: X linked; Het: Heterozygous; Hom: Homozygous; Hem: Hemizygous; OMIM: Online
Mendelian Inheritance in Man ™.

| | S | | | | | | | | |
|---|---|---|---|---|---|---|---|---|---|
| | or | | Affected | OMIM | Inher- | | de novo or | | |
| ID | T | Disease | Gene | ID | itance | Zygosity | imherited | Variant 1 (V1) | Variant 2 (V2) |
| 1 | S | Hereditry Fructose | ALDOB | 229600 | AR | Het | Unknown | c.448G > C, | c.524C > A, |
| | | Intelorance | | | | | | p.Ala150Pro | p.Ala175Asp |
| 2 | S | Ornithine | OTC | 311250 | SL | Hem | DeNovo | c.275G > A | |
| | | Transcarbamylase | | | | | | p.Arg92Gln | |
| | | Deficiency | | | | | | | |
| 3 | S | Propionic | PCCA | 606054 | AR | Hom | Unknown | c.1899 + 4_1899 + 7 | |
| | | Acidemia | | | | | | del | |
| 4 | T | Developmental and | SCNA | 613721 | AD | Het | DeNovo | c.4437G > C, | |
| | | epileptic | | | | | | p.Gln1476His | |
| | | envephalopathy, | | | | | | | |
| | | type 11 | | | | | | | |
| | | | | | V1 | V2 | Age at | | |
| | | | | | P/ | P/ | enrollment | | Consan- |
| | | | | ID | LP | LP | (days) | Sex | guinity |
| | | | | 1 | P | P | 107 | [symbol] | N |
| | | | | 2 | P | | 5 | [symbol] | N |
| | | | | 3 | LP | | 4 | [symbol] | N |
| | | | | 4 | LP | | 7 | [symbol] | N |

TABLE 12
Analytic performance of three automated interpretation software systems, MOON ™ (InVitae), GEM ™ (Fabric Genomics) and
TruSight ™ (Illumina) in four retrospective cases and one prospective case. * Includes processing time for DRAGEN ™
v3.7. Abbreviations: SNV: single nucleotide variant; SV: structural variant; CNV: copy number variant.

| Case Number | AG928 | AI115 | AI148 | AI185 | Average | AH638 |
|---|---|---|---|---|---|---|
| Run | 1020 | 1204 | 1208 | 1218 | | 1026 |
| Type of case | | Retrospective | | | | Prospective |
| Diagnosis | ALDOB | OTC | PCCA | SCN2A | | SLC19A3 |
| MOON (InVitae) | 1 | 1 | 1 | 1 | 1 | 1 |
| Rank of correct diagnosis | | | | | | |
| SNVs | 6 | 7 | 9 | 4 | 6.5 | 10 |
| SV/CNVs | 20 | 1 | 11 | 8 | 10 | 0 |
| Total variants | 26 | 8 | 20 | 12 | 16.5 | 10 |
| Time to provisional diagnosis (min) | 9 | 10 | 12 | 10 | 10.25 | 10 |
| GEM (Fabric Genomics) | 3 | 1 | 1 | 4 | 2.25 | 1 |
| Rank of correct diagnosis | | | | | | |
| Total variants (including SV/CNVs) | 5 | 6 | 5 | 16 | 8 | 8 |
| Time to provisional diagnosis (min) | 39 | 43 | 44 | 40 | 41.5 | 48 |
| Trusight Software Suite (Illumina) | 1 | 1 | 1 | 1 | 1 | 1 |
| Rank of correct diagnosis | | | | | | |
| SNVs | 5 | 2 | 2 | 5 | 3.5 | 15 |
| SV/CNVs | n.a. | n.a. | n.a. | n.a. | n.a. | n.a. |
| Total variants | 5 | 2 | 2 | 5 | 3.5 | 15 |
| Time to provisional diagnosis (min)* | 213 | 230 | 178 | 276 | 224.25 | 220 |

Although the invention has been described with reference to the above examples, it will be understood that modifications and variations are encompassed within the spirit and scope of the invention. Accordingly, the invention is limited only by the following claims.

What is claimed is:

1. A method comprising:
a) determining a phenome of a subject from an electronic medical record (EMR), wherein the phenome comprises a plurality of clinical phenotypes extracted from the EMR;
b) translating the clinical phenotypes into a standardized vocabulary;
c) generating a first list of potential differential diagnoses of the subject, the first list optionally being rank ordered;
d) performing genetic sequencing of a DNA sample from the subject;
e) determining genetic variants of the DNA;
f) analyzing the results of (c) and (e) to generate a second list of potential differential diagnoses of the subject, the second list being rank ordered;
g) determining the efficacy and/or quality of evidence of efficacy of available treatments for the second list of potential differential diagnoses;
h) analyzing the results of (f) and (g) to generate a third list of potential differential diagnoses of the subject, the third list being rank ordered, together with available treatments; and
i) generating a report comprising results of any of (a)-(h).

2. The method of claim 1, further comprising generating the EMR for the subject prior to (a).

3. The method of claim 1, wherein (b) utilizes natural language processing to perform the translation.

4. The method of claim 1, wherein (a)-(c) and (d)-(e) are performed in parallel.

5. The method of claim 1, wherein genetic sequencing comprises, genome sequencing, rapid whole genome sequencing (rWGS), ultra-rapid whole genome sequencing, exome sequencing, or rapid whole exome sequencing (rWES).

6. The method of claim 5, wherein the DNA sample is from a biological sample.

7. The method of claim 6, wherein the sample is blood, dried blood spot, serum, saliva, buccal smear/swab, plasma, feces, cerebrospinal fluid or urine.

8. The method of claim 1, wherein the first, second and/or third ranked list is generated via query of a database populated with known clinical phenotypes of all known genetic diseases expressed in the same vocabulary as the standardized vocabulary of (b).

9. The method of claim 1, wherein determining genetic variants of (e) further comprises annotation and classification of pathogenicity of the genetic variants.

10. The method of claim 9, wherein the genetic variants are utilized to generate a probabilistic diagnosis and/or are annotated and classified as being of uncertain significance (VUS), pathogenic (P) or likely pathogenic (LP).

11. The method of claim 9, wherein only genetic variants with an allele frequency of <5%, 2.5%, 1%, 0.1% or less in a population of healthy individuals is retained.

12. The method of claim 11, wherein determining genetic variants of (e) further comprises annotation of the genetic variants to identify and rank all diplotypes as being of uncertain significance (VUS), pathogenic (P) or likely pathogenic (LP) on the basis of pathogenicity.

13. The method of claim 12, wherein the second list of potential differential diagnoses is generated by comparing the annotated VUS, LP and P diplotypes on a regional genomic basis with corresponding genomic regions associated with the first list of potential differential diagnoses of (c).

14. The method of claim 13, wherein the genetic variants are ranked based on a combination of rank of goodness of fit of clinical phenotypes, rank of pathogenicity of diplotypes, and/or allele frequencies of the genetic variants in a population of health individuals.

15. The method of claim 1, wherein (h) further comprises annotation and classification of the available treatments.

16. The method of claim 15, wherein the available treatments are utilized to generate a probabilistic diagnosis.

17. The method of claim 15, wherein the available treatments are annotated and classified as being safe and effective (SE), safe but with little evidence of effectiveness (SmodE), moderate risk and effective (modSE), moderate risk but with little evidence of effectiveness (modSmodE), high risk and effective (highRE), or high risk and with little evidence of effectiveness (highRmodE); the available treatments include drug, dietary, device and surgical interventions; and/or the available treatments include modified code status or palliative care or comfort care.

18. The method of claim 15, wherein the third list of potential differential diagnoses is generated by comparing the second list of potential differential diagnoses corresponding to genomic regions associated with the first list of potential differential diagnoses of (c).

19. The method of claim 1, further comprising: j) determining the availability of confirmatory tests for the third list of potential differential diagnoses; k) analyzing the results of (g) and (h) to generate a fourth list of potential differential diagnoses of the subject, the fourth list being rank ordered, together with available confirmatory tests; and/or generating a report comprising results of any of (j)-(k).

20. The method of claim 1, wherein genetic sequencing is performed for both biological parents and only results in which trio diplotypes fit a known inheritance pattern of a specific genetic disease are obtained.

21. The method of claim 20, wherein genetic sequencing is performed for both biological parents, wherein parental health status (healthy or affected) is used to obtain only results in which parental diplotypes fit a known inheritance pattern of a specific genetic disease.

22. The method of claim 21, wherein genetic variants present in the subject's genome and not in the parental genome are utilized to determine a diagnosis for the subject.

23. The method of claim 1, wherein the subject is less than 5 years old.

24. The method of claim 22, wherein the subject is an infant, fetus or neonate.

25. The method of claim 1, wherein the potential differential diagnoses comprise genetic diseases.

26. The method of claim 1, wherein the method is automated.

27. The method of claim 1, further comprising generating a therapy regime for the subject and/or providing a therapy to the subject.

28. The method of claim 27, wherein the potential differential diagnoses comprise cancer.

29. The method of claim 28, wherein the therapy is selected from the group consisting of surgery, adjuvant chemotherapy, neoadjuvant chemotherapy, radiation therapy, hormone therapy, cytotoxic therapy, immunotherapy, adoptive T cell therapy, targeted therapy, or any combinations thereof.

30. The method of claim 1, wherein (a) further comprises analyzing supplemental clinical information to determine the phenome.

31. The method of claim 1, wherein (a) is performed for a plurality of subjects thereby generating a plurality of EMRs, a plurality of phenomes, and a plurality of clinical phenotypes.

32. The method of claim 2, wherein (a) is performed for a plurality of subjects thereby generating a plurality of EMRs, a plurality of phenomes, and a plurality of clinical phenotypes.

33. The method of claim 32, further comprising storing on a non-transitory memory the plurality of EMRs, the plurality of phenomes, and the plurality of clinical phenotypes to generate a searchable database.

34. The method of claim 33, further comprising utilizing the database to screen for genetic data, a genotype, or a disease or disorder in a second subject or to update a diagnosis of the subject.

35. The method of claim 1, wherein one or more of (a)-(k) are adjustable by a user to determine available diagnoses and available treatments based on the available diagnoses to provide dynamic treatment to the subject.

36. A system comprising:
a controller including at least one processor and non-transitory memory, wherein the controller is configured to perform any one, or combination of (a)-(k) of claim 1.
